(19) United States
(12) Patent Application Publication
(10) Pub. No.: US 2019/0164632 A1
(43) Pub. Date: May 30, 2019

(54) DRUG INDICATION AND RESPONSE PREDICTION SYSTEMS AND METHOD USING AI DEEP LEARNING BASED ON CONVERGENCE OF DIFFERENT CATEGORY DATA

(71) Applicant: SYNTEKABIO CO., LTD., Daejeon (KR)

(72) Inventors: Jongsun JUNG, Daejeon (KR); Yoosup CHANG, Namyangju-si (KR); Hyejin PARK, Yongin-si (KR); Seung-Ju LEE, Daegu (KR); Jae-Min SHIN, Yongin-si (KR)

(73) Assignee: SYNTEKABIO CO., LTD., Daejeon (KR)

(21) Appl. No.: 16/198,138

(22) Filed: Nov. 21, 2018

(30) Foreign Application Priority Data
Sep. 25, 2017 (KR) 10-2017-0123719
Dec. 31, 2017 (KR) 10-2017-0185040

(51) Int. Cl. G16B 40/30; G16C 20/70; G06N 3/08; G06N 3/04

(52) U.S. Cl. CPC G16B 40/30; G06N 3/04; G06N 3/08; G16C 20/70

(57) ABSTRACT

A system of predicting drug indications and drug response using an artificial intelligence (AI) deep learning model based on convergence of different types of information, the system including: a learning module configured to learn the response correlation between structure information on a drug and genetic information on a genome from collected learning information by deep machine learning; a prediction module configured to receive analysis information and output the result of prediction of the response of the genome to the drug from the analysis information; and a storage module configured to store a response prediction algorithm learned by the learning module. The learning information is drug response information obtained from clinical drug response information on target proteins, cell lines or living bodies.

### CROSS REFERENCE TO PRIOR APPLICATIONS

This application claims priority to and the benefit of Korean Patent Application Nos. 10-2017-0123719 filed on Sep. 25, 2017 and 10-2017-0185040 filed on Dec. 31, 2017, the disclosure of which are incorporated herein by reference in their entirety.

### BACKGROUND

The present invention relates to cancer-drug response scanning (CDRscan), which is used for a system and a method for predicting drug indications and drug response and is a novel learning model capable of reliably predicting drug response by analyzing the convergence of specific genetic variation fingerprints associated with diseases, including cancers, and the molecular profiles of drugs.

Recently, the evolution of next generation sequencing (NGS) technologies has made many advances in understanding complex and various cancers. Moreover, due to international consortium efforts, not only the catalogue of somatic mutations in these cancers, but also a comprehensive database of cancer driver mutations have been developed and published [Non-Patent Documents 1, 2 and 3]. Due to the results of this international consortium study, expectations for cancer-specific therapies for specific genomic fingerprints of individual tumors have also increased rapidly. However, there is still not enough new personalized cancer treatments which are approved and used clinically for all stakeholders in the medical community, including cancer patients and the pharmaceutical industry [Non-Patent Document 4]. Therefore, an efficient and systematic approach is needed to predict the personalization relationship between genomic information and anticancer drug responses.

Several collaborative efforts have been made to integrate molecular profiling data on cancer cell lines and drug toxicity data (www.lincsproject.org) [Non-Patent Documents 5 and 6]. The most important goal of these efforts is to identify genomic biomarkers that can predict anticancer drug toxicity and personalized drugs.

Of the genotoxicity information on drug toxicity in cancer, GDSC (Genomics of Drug Sensitivity in Cancer) is an example of a publicly available database (cancerRxgene.org). In particular, GDSC is a public database providing experimentally measured drug sensitivities of 1,001 human cancer cell lines against 265 anticancer compounds [Non-Patent Document 6]. The GDSC cell line project (CCLP: COSMIC Cell Lines Project) used here was published at http://cancer.sanger.ac.uk/cell lines. These common resources are expected to be of great help in realizing genome-based precision cancer treatments. However, despite the potential value of these databases, the high dimensionality and complexity of the data poses problems for integrative analysis. Thus, many computational methods have been developed to systematically characterize molecular biomarkers in anticancer drug toxicity [Non-Patent Documents 5, 7, 8, 9, 10, 11, 12 and 13]. Despite these efforts, drug toxicity is limited to certain cell lines and a given set of gene mutations. This is because everybody's genetic information is completely different between people and common mutations are part of the whole.

With the recent advances in in information technology, methods, called deep learning models or in-depth learning models, have been more and more commonly used to solve the above-mentioned complexity [Non-Patent Document 14]. The deep learning method is a branch of technology based on deep machine learning from a large volume of high-dimensional raw data [Non-Patent Document 15]. Until recently, the efficacy of learning was directly limited to the availability of relevant data [Non-Patent Document 16]. Nevertheless, with a methodological improvement and a powerful machine with parallel computing horsepower, a deep learning model can be trained with multiple hidden layers, containing thousands of hidden units [Non-Patent Documents 17, 18, 19 and 20].

Since it can operate several types of structural information, such as pharmacological, genomic, transcriptomic and epigenomic data and their drug response data, it is suitable for predicting drug-target interaction with minimal guidance [Non-Patent Document 14].

The pharmaceutical industry has begun showing its vested interest in deep learning to exploit these types of data for new drug development [Non-Patent Document 21]. Recently, several promising results have been demonstrated using deep learning in drug development [Non-Patent Documents 22, 23, 24 and 25]. In addition, drug-target profiling [Non-Patent Document 26] and drug repositioning with superior prediction accuracy compared to other conventional machine learning models [Non-Patent Document 27] became possible. However, the majority of the approaches have just proven the concept, and there is now a shortage of possible solutions for drug discovery through deep learning [Non-Patent Document 28].

Currently, PubChem (pubchem.ncbi.nlm.nih.gov) is run by the National Center for Technology Information (NCBI) and covers about 100 million compounds, 200 million substances and bioassay information (en.wikipedia.org/wiki/PubChem). There are also many methods that express such compounds as pharmacophore descriptors [Non-Patent Documents 29, 30, 31, 32 and 33]. Among them, the PaDELL method can express 1,875 features (1, 444 1D and 2D, and 431 3D) and 12 fingerprints (about 16,092 bits overall) in the drug [Non-Patent Document 29]. Moreover, variations in genomes can extract various features. In particular, methods and tools for extracting mutations that cause diseases are as described in Non-Patent Documents 34 to 56.

Therefore, in the prior art, quantitative structure activity relationship (QSAR), drug development using drug cytotoxicity data, regulation of expression of deep learning-based whole genome sequencing, structural variation and the like were independently applied. However, in the present invention, CDRscan (cancer drug response scanning), which is an AI deep-learning method that integrates different types of feature information (genomic information, QSAR information, and expression information) into drugs-cell lines-toxicity (IC50) data has improved predictive accuracy compared to previous computer modeling approaches. In particular, a model of interaction of virtual drugs vs. cell lines or target proteins is shown in FIG. 1. Of the two different types of virtual information, the first information (drug information) is explained by the PaDELL method or the documents [Non-Patent Documents 29 to 33]. In addition, the second information can be explained by the document methods [Non-Patent Documents 34 to 56] for the genomic fingerprint (or a set of mutation features) of the full-length genome, and the most standard deep learning method is given in the document [Non-Patent Document 57]. The method of the present invention can be used for an accurate drug response prediction model and a clinical decision supporting system for drug repurposing/repositioning, chemical screening, identification of new anticancer drug candidates, and selection of patient-specific anticancer drugs.

Meanwhile, the following non-patent prior art documents are classified as follows according to main contents.

(001 to 004) are papers on the relationship between genomic information and the response of anticancer drugs;

(005 to 13) are references to cancer genomic drug toxicity and the COSMIC cell line project; (014 to 018) are pharmacology- and genome-related papers on deep learning models;

(019 to 028) are papers used in new drug development for deep learning models;

(029 to 056) are methods and articles that express drugs and variations as features;

(057) is a paper on deep learning methodology and algorithm.

### PRIOR ART DOCUMENTS

### Non-Patent Documents

(Non-Patent Document 1) Forbes, S. A., et al. COSMIC: somatic cancer genetics at high-resolution. Nucleic Acids Research. 45, 777-783 (2016).
(Non-Patent Document 2) Lawrence, M. S., et al. Discovery and saturation analysis of cancer genes across 21 tumour types. Nature. 505, 495-501 (2014).
(Non-Patent Document 3) Stratton, M. R., Campbell, P. J. & Futreal, P. A. The cancer genome. Nature 458, 719-724 (2009).
(Non-Patent Document 4) Williams S P, & McDermott U. The pursuit of therapeutic biomarkers with high-throughput cancer cell drug screens. Cell Chemical Biology. 24, 1066-1074 (2017).
(Non-Patent Document 5) Barretina, J, et al. The Cancer Cell Line Encyclopedia enables predictive modelling of anticancer drug sensitivity. Nature. 483, 603-7 (2012).
(Non-Patent Document 6) Yang, W., et al. Genomics of Drug Sensitivity in Cancer (GDSC): a resource for therapeutic biomarker discovery in cancer cells. Nucleic Acids Research. 41, 955-961 (2012).
(Non-Patent Document 7) Basu, A., et al. An interactive resource to identify cancer genetic and lineage dependencies targeted by small molecules. Cell. 154, 1151-1161 (2013).
(Non-Patent Document 8) Iorio, F., et al. (2016). A Landscape of pharmacogenomic interactions in cancer. Cell. 166, 740-754 (2016).
(Non-Patent Document 9) Garnett, M. J., Edelman, E. J., Heidorn, S. J., Greenman, C. D., Dastur, A., Lau, K. W., Greninger, P., Thompson, I. R., Luo, X. & Soares, J. Systematic identification of genomic markers of drug sensitivity in cancer cells. Nature. 483, 570-575 (2012).
(Non-Patent Document 10) Menden, M. P., Iorio, F., Ballester, P. J., Saez-Rodriguez, J., Garnett, M., McDermott, U., & Benes, C. H. Machine learning prediction of cancer cell sensitivity to drugs based on genomic and chemical properties. PLoS ONE. 8. e61318 (2013).
(Non-Patent Document 11) Rubio-Perez, C., Tamborero, D., Schroeder, M., Antolin, A., Deu-Pons, J., Perez-Llamas, C., Mestres, J., Gonzalez-Perez, A., & Lopez-Bigas, N. In silico prescription of anticancer drugs to cohorts of 28 tumor types reveals targeting opportunities. Cancer Cell. 27, 382-396 (2015).
(Non-Patent Document 12) Seashore-Ludlow, B., et al. Harnessing connectivity in a large-scale small-molecule sensitivity dataset. Cancer Discovery. 5, 1210-1223 (2015).
(Non-Patent Document 13) Yadav, B., et al. Quantitative scoring of differential drug sensitivity for individually optimized anticancer therapies. Scientific Reports. 4 (2014).
(Non-Patent Document 14) Vanhaelen, Q., et al. Design of efficient computational workflows for in silico drug repurposing. Drug Discovery Today. 22, 210-222 (2016).
(Non-Patent Document 15) Mamoshina, P., Vieira, A., Putin, E. & Zhavoronkov, A. Applications of deep learning in biomedicine. Molecular Pharmaceutics. 13, 1445-1454 (2016).
(Non-Patent Document 16) Ramsundar, B., Kearnes, S., Riley, P., Webster, D., Konerding, D. & Pande, V. Massively multitask networks for drug discovery. arXiv:1502.02072 (2015).
(Non-Patent Document 17) Dahl, G. E., Jaitly, N. Salakhutdinov, R. Multi-task neural networks for QSAR predictions. arXiv:1406.1231 (2014).
(Non-Patent Document 18) Nantasenamat C, Isarankura-Na-Ayudhya C, Naenna T, Prachayasittikul V. “A practical overview of quantitative structure-activity relationship”. Excli J. 8: 7488 (2009).
(Non-Patent Document 19) Ebuka, D Quantitative structure activity relationship study on potent anticancer compounds against MOLT-4 and P388 leukemia cell lines, Journal of Advanced Research, 10.1016 (2016)
(Non-Patent Document 20) Yuan, Y., et al. DeepGene: an advanced cancer type classifier based on deep learning and somatic point mutations. BMC Bioinformatics. 17, 243-256 (2016).
(Non-Patent Document 21) Smalley, E. AI-powered drug discovery captures pharma interest. Nature Biotechnology. 35, 604-605 (2017).)
(Non-Patent Document 22) Baskin, I. I., Winkler, D. & Tetko, I. V. A renaissance of neural networks in drug discovery. Expert Opinion on Drug Discovery. 11, 785-95 (2016).
(Non-Patent Document 23) Gonczarek, A., Tomczak, J. M., Zareba, S., Kaczmar, J. Dabrowski, P. & Walczak, M J. Learning deep architectures for interaction prediction in structure-based virtual screening. NIPS, 30, (2017).
(Non-Patent Document 24) Pereira, J. C., Caffarena, E. R., & Dos Santos, C. N. Boosting docking-based virtual screening with deep learning. Journal of Chemical Information and Modeling. 56, 2495-2506 (2016).
(Non-Patent Document 25) Unterthiner, T, Mayr, A, Klambauer, G, Steijaert, M, Wegner, J. K., Ceulemans, H, & Hochreiter, S. Deep learning as an opportunity in virtual screening. NIPS, 27, (2014).
(Non-Patent Document 26) Wen M., Zhang Z., Niu S., Sha H., Yang R., Lu H., & Yun Y. Deep-learning-based drug-target interaction prediction. Journal of Proteome Research. 16, 1401-1409 (2017).
(Non-Patent Document 27) Aliper A, Plis S, Artemov A, Ulloa Mamoshina P, & Zhavoronkov A. Deep learning applications for predicting pharmacological properties of drugs and drug repurposing using transcriptomic data. Molecular Pharmaceutics. 13, 2524-2530 (2016).
(Non-Patent Document 28) Ching, T., et al. Opportunities and obstacles for deep learning in biology and medicine. bioRxiv. doi: http://dx.doi.org/10.1101/142760 (2017).
(Non-Patent Document 29) Yap C W. PaDEL-Descriptor: An open source software to calculate molecular descriptors and fingerprints. Journal of Computational Chemistry. 32, 1466-1474 (2010)
(Non-Patent Document 30) Schneider, G.; Clement-Chomienne, O.; Hilfiger, L.; Schneider, P.; Kirsch, S.; Bohm, H-J. and Neihart, W. Virtual Screening for Bioactive Molecules by Evolutionary De Novo Design Angew. Chem. Int. Ed., 39, 4130-4133 (2000)
(Non-Patent Document 31) Schneider, G.; Lee, M-L.; Stal, M. and Schneider, P. De novo design of molecular architectures by evolutionary assembly of drug-derived building blocks J. Comp-Aid. Mol. Des., 14, 487-494 (2000)
(Non-Patent Document 32) Pearlman, S. R. and Smith, K. M. Novel Software Tools for Chemical Diversity, Perspectives in Drug Discovery and Design, 9/10/11: 339-353, (1998).
(Non-Patent Document 33) Burden, F. R. Molecular identification number for substructure searches, J. Chem. Inf. Comput. Sci. 29, 225-7 (1989).
(Non-Patent Document 34) SIFT: Kumar, Prateek, Steven Henikoff, and Pauline C. Ng. “Predicting the effects of coding non-synonymous variants on protein function using the SIFT algorithm.” Nature protocols 4.7: 1073-1081 (2009).
(Non-Patent Document 35) Polyphen-2: I. A. Adzhubei, S. Schmidt, L. Peshkin et al., method and server for predicting damaging missense mutations, Nature Methods, vol. 7, no. 4, pp. 248249, 2010
(Non-Patent Document 36) LRT S. Chun and J. C. Fay, of deleterious mutations within three human genomes, Genome Research, vol. 19, no. 9, pp. 15531561, 2009.
(Non-Patent Document 37) Polyphen-2 HDIV n HDVAR Score: Yunos, R. I. M., Ab Mutalib, N. S., Khor, S. S., Saidin, S., Nadzir, N. M., Razak, Z. A., & Jamal, R. (2016). Characterisation of genomic alterations in proximal and distal colorectal cancer patients (No. e2109v1). PeerJ Preprints.
(Non-Patent Document 38) MutationAccessor1: Reva, B., Antipin, Y., & Sander, C. (2011). Predicting the functional impact of protein mutations: application to cancer genomics. Nucleic acids research, 39(17), e118-e118.
(Non-Patent Document 39) Mutation Accessor2: Gnad, F., Baucom, A., Mukhyala, K., Manning, G., & Zhang, Z. Assessment of computational methods for predicting the effects of missense mutations in human cancers. BMC genomics, 14(3), S7 (2013).
(Non-Patent Document 40) MUTATIONTASTER: Dong, C., Wei, P., Jian, X., Gibbs, R., Boerwinkle, E., Wang, K., & Liu, X. Comparison and integration of deleteriousness prediction methods for nonsynonymous SNVs in whole exome sequencing studies. Human molecular genetics, 24(8), 2125-2137 (2014).
(Non-Patent Document 41) Mutation Accessor and Mutation Taster: Oishi, Maho, et al. “Comprehensive Molecular Diagnosis of a Large Cohort of Japanese Retinitis Pigmentosa and Usher Syndrome Patients by Next-Generation Sequencing Diagnosis of RP and Usher Syndrome Patients by NGS.” Investigative ophthalmology & visual science 55.11 (2014): 7369-7375.
(Non-Patent Document 42) PhyloP46way_placental and PhyloP46way_vertebrate: Pollard, Katherine S., et al. “Detection of nonneutral substitution rates on mammalian phylogenies.” Genome research 20.1: 110-121 (2009).
(Non-Patent Document 43) GERP++_RS Score: Davydov, E. V., Goode, D. L., Sirota, M., Cooper, G. M., Sidow, A., & Batzoglou, S. Identifying a high fraction of the human genome to be under selective constraint using GERP++. PLoS computational biology, 6(12), e1001025 (2010).
(Non-Patent Document 44) B62 Score: Tsuda, H., Kurosumi, M., Umemura, S., Yamamoto, S., Kobayashi, T., & Osamura, R. Y. HER2 testing on core needle biopsy specimens from primary breast cancers: interobserver reproducibility and concordance with surgically resected specimens. BMC cancer, 10(1), 534 (2010).
(Non-Patent Document 45) Siphy: Garber, Manuel, et al. “Identifying novel constrained elements by exploiting biased substitution patterns.” Bioinformatics 25.12: i54-i62 (2009).
(Non-Patent Document 46) CHASM: H. Carter, J. Samayoa, R. H. Hruban, and R. Karchin, of driver mutations in pancreatic cancer using cancerspecific high-throughput annotation of somatic mutations (CHASM), Cancer Biology & Therapy, vol. 10, no. 6, pp. 582587 (2010).
(Non-Patent Document 47) Dendrix: F. Vandin, E. Upfal, and B. J. Raphael, novo discovery of mutated driver pathways in cancer, Genome Research, vol. 22, no. 2, pp. 375385 (2011).
(Non-Patent Document 48) MutsigCV: M. S. Lawrence, P. Stojanov, P. Polak et al., heterogeneity in cancer and the search for new cancer-associated genes, Nature, vol. 499, no. 7457, pp. 214218. [68] M. Kanehisa and S. Goto, kyoto encyclopedia (2013)
(Non-Patent Document 49) FATHMM: Shihab, Hashem A., et al. “Predicting the functional, molecular, and phenotypic consequences of amino acid substitutions using hidden Markov models.” Human mutation 34.1: 57-65 (2013).
(Non-Patent Document 50) VEST3 score: Carter, Hannah, et al. “Identifying Mendelian disease genes with the variant effect scoring tool.” BMC genomics 14.3: S3 (2013).
(Non-Patent Document 51) MetaSVM: Nono, Djotsa, et al. “Computational Prediction of Genetic Drivers in Cancer.” eLS (2016).
(Non-Patent Document 52) MetaLR: Dong, Chengliang, et al. “Comparison and integration of deleteriousness prediction methods for nonsynonymous SNVs in whole exome sequencing studies.” Human molecular genetics 24.8: 2125-2137 (2014).
(Non-Patent Document 53) CADD: Kircher, Martin, et al. “A general framework for estimating the relative pathogenicity of human genetic variants.” Nature genetics 46.3: 310-315 (2014).
(Non-Patent Document 54) CADD 2: Velde, K. Joeri, et al. “Evaluation of CADD scores in curated mismatch repair gene variants yields a model for clinical validation and prioritization.” Human mutation 36.7: 712-719 (2015).
(Non-Patent Document 55) CADD 3: Mather, Cheryl A., et al. “CADD score has limited clinical validity for the identification of pathogenic variants in non-coding regions in a hereditary cancer panel.” Genetics in medicine: official journal of the American College of Medical Genetics (2016).
(Non-Patent Document 56) ParsSNP: Kumar, Runjun D., S. Joshua Swamidass, and Ron Bose. “Unsupervised detection of cancer driver mutations with parsimony-guided learning.” Nature genetics 48.10: 1288-1294 (2016).
(Non-Patent Document 57) Deep Learning: Yann Lecun, Y., Bengio, Y. & Hinton, G. Nature. 521, 436-444 (2015).

### SUMMARY

The present invention has been made in accordance with the technical background and societal requirements as described above, and is intended to provide a system for predicting drug indications and drug response, which is used to predict drug response based on the genetic features and fingerprints of a target genome. A specific object of the present invention is to provide a prediction system which is capable of reliably predicting the response between structure information on drugs and the specific genetic variations or fingerprints of genomes, from drug response results for known clinical drug response data on cell line genomes, target proteins and living bodies, through deep machine learning.

The present invention has been made in order to solve the above-described problems occurring in the prior art, and a system for predicting drug indications and drug response comprises: a learning module configured to learn the response correlation between structure information on a drug and genetic information on a genome from collected learning information by deep machine learning; a prediction module configured to receive analysis information and output the result of prediction of the response of the genome to the drug from the analysis information; and a storage module configured to store a response prediction algorithm learned by the learning module, wherein the learning information is drug response information obtained from clinical drug response data on cell line genomes, target proteins and living bodies.

Here, the learning module may comprise: a learning data generation unit configured to generate learning data for deep machine learning from the collected learning information; a deep machine learning unit configured to perform deep machine learning for a plurality of learning data generated from the learning data generation unit; and a response prediction algorithm generation unit configured to predict the response of the genome to the drug.

The drug information may be information on nutrients, unspecified drugs (whose toxicity is not known), or specified drugs (FDA-approved drugs). Furthermore, the drug information may be defined as information on region a) in FIG. 2.

The structure information may be descriptor information on the drug. In addition, the structure information on the drug may be defined as information of region d) in FIG. 2.

The genetic information may be mutation information on the genome.

In addition, the genetic information may also be feature information on mutations contained in the genome.

The feature information may be genomic fingerprints for the mutations and may comprise any one or more of mutability or entropy of variants, variant frequency in cancer, driver mutation score, 3D structure mutation environment, clinical significance mutation, drug response stratification attributable to genetic interaction, epigenomics, transcriptomics, and proteomics. In addition, the genetic feature formation may be defined as information of region e) in FIG. 2.

The learning data may be a plurality of information that represent the response between a group of mutation information contained in the target protein, cell line genome and drug response clinical information and a group of descriptor information on the drug. In addition, the learning data may be defined as information of region c) in FIG. 2.

In addition, the learning data may also be a plurality of information that represent the drug indications/response between a group of genetic feature information on mutations contained in the cell line genomes and a group of descriptor information on the drugs.

The deep machine learning unit may be configured to learn the response correlation between each genetic information on the cell lines and each structure information on the drugs by deep machine learning for the learning data.

In addition, the deep machine learning may also be performed by a CNN (Convolutional Neural Network) model.

Furthermore, the deep machine learning may also be performed by a TensorFlow machine learning engine.

In addition, the learning information may be collected from target protein-drug dissociation constant, cancer cell line encyclopedia (CCLE), or genomics of drug sensitivity in cancer (GDSC), or in vivo experimental databases.

In addition, the learning information may also be collected from databases including target protein-drug dissociation constant (Kd) and genetic information.

In addition, the learning information may also be collected from in vivo drug response databases for genetic information-based patients with personalized drug prescriptions collected from hospitals (or clinical drug experiments).

The deep machine learning may comprise the steps of: (A1) collecting learning information which represents the response of each cell line genome to each drug; (A2) generating genetic information on genomes from the learning information; (A3) generating structure information on the drug from the learning information; (A4) generating learning layers that represent the response between a group of the genetic information on the genomes and a group of the structure information on the drugs from the learning information; and (A5) deriving the response correlation between individual genetic information and individual structure information by deep machine learning for the learning layers.

In addition, the response may be determined by the drug dissociation constant of the target protein, the inhibition index IC₅₀ of the cell line, or anticancer drug treatment effects (complete remission (CR), partial remission (PR), stable disease (SD), or progressive disease (PD)) in patients.

The response prediction algorithm generation unit may be configured to generate an algorithm that generates the response between genetic information on the genome and structure information on the drug, through the response correlation between the genetic information and the structure information, learned by the deep machine learning unit.

Furthermore, the prediction of drug response by the prediction module may comprise the steps of: (C1) receiving analysis information;

(C2) generating genetic information for analysis on genomes from the analysis information; (C3) generating structure information for analysis on drugs from the analysis information; and (C4) outputting the result of prediction of the response of the genome to the drug from the analysis information on the basis of the response correlation between the genomic information for analysis and the structure information for analysis by the response prediction algorithm.

The structure information for analysis may be descriptor information on the drug.

The genetic information for analysis may be mutation information on the genome.

In addition, the genetic information for analysis may also be feature information on mutations contained in the genome.

The prediction algorithm may be configured to merge prediction values generated by different deep machine learning prediction algorithms.

The different deep machine learning prediction algorithms may be configured to apply a Convolutional Neural Network (CNN) model in independent layers of each of different types of information, then generate a layer in which different types of information are fully connected, then calculate the weighted sum of hidden units, then apply nonlinear function Relu, hyperbolic tangent, sigmoid function, or new function with improved performance provided in TensorFlow, to the calculation results.

Meanwhile, the deep machine learning may comprise the steps of: (B1) collecting learning information that represents the response of each cell line genome to each drug;

(B2) generating genetic information on genomes contained in the learning information; (B3) generating genetic information learning layers that represent the response between a group of the genetic information on each genome and the drug; (B4) generating the response correlation between each genetic information and the drug by deep machine learning for the genetic information learning layers; (B5) generating structure information on the drug contained in the learning information; (B6) generating structure information learning layers that represent the response between each genome and a group of the structure information on the drug; (B7) generating the response correlation between each genome and each structure information by deep machine learning for the structure information learning layers; and (B8) generating the response correlation between individual genetic information and individual structure information through the response correlation between each genetic information and the drug, generated in step (B4), and the response correlation between each genome and each structure information, generated in step (B7).

### BRIEF DESCRIPTION OF THE DRAWINGS

FIG. 1 illustrates one example of the deep machine learning structure of a CDRscan according to the present invention.

FIG. 2 is a block diagram showing the configuration of a drug indication and drug response prediction system of the present invention, divided according to function.

FIG. 3 is a flow chart showing one example of a deep machine learning method which embodies a drug indication and drug response prediction method of the present invention.

FIG. 4 is a flow chart showing another example of a deep machine learning method which embodies a drug indication and drug response prediction method of the present invention.

FIG. 5 is a flow chart showing one example of a drug response prediction method which embodies a drug indication and drug response prediction method of the present invention.

FIG. 6 illustrates drug information, genetic information, and information on the responsiveness and features thereof for deep machine learning according to the present invention.

FIG. 7 illustrates one example of a PeDEL pharmacophore descriptor according to the present invention.

FIG. 8 illustrates one example of a process for generating IC_{H} data for a drug which is applied in the present invention.

FIG. 9 illustrates one example of the configuration of a process for generating genetic information on a cell line according to the present invention.

FIG. 10 illustrates a structure for generating data on the relationship between disease-related genome and drug toxicity, which is used in the present invention.

FIG. 11 illustrates a process for generating data on the relationship between disease-related genome and drug toxicity according to the present invention.

FIG. 12 illustrates an example of each step of a deep machine learning method according to the present invention.

FIG. 13 illustrates one example of the convergence of different types of information for deep machine learning according to the present invention.

FIG. 14 shows cell line-based drug toxicity experiment data and drug response prediction results according to the present invention.

FIG. 15 shows the results of predicting drug binding affinity based on a target protein according to the present invention and drug binding affinity by simulation.

FIG. 16 illustrates simulations and drug interaction energy data sources for calculation of target protein-drug binding affinity according to the present invention.

FIG. 17 illustrates drug interaction energy data sources for calculation of target protein-drug binding affinity according to the present invention.

FIG. 18 illustrates mutation features, DNA flanking sequences and protein flanking sequences.

FIG. 19 illustrates experiments which embody in vitro and in vivo drug indication and response prediction methods according to the present invention.

FIG. 20 illustrates correlation (R²) values for the drug indication and drug response prediction results according to the present invention.

FIG. 21 illustrates the results of obtaining correlation (R²) values for the drug indication and drug response prediction results for each cell line according to the present invention.

FIG. 22 illustrates the results of obtaining correlation (R²) values for the drug indication and drug response prediction results for each drug according to the present invention.

FIG. 23 shows the results of predicting new applications of conventional drugs according to the present invention.

FIG. 24 shows the result of generating an ROC-curve for the accuracy of a prediction model in which different types of feature information are merged according to the present invention.

FIG. 25 illustrates the results of obtaining R² values for individual cancer types by a prediction model in which different types of feature information are merged according to the present invention.

FIG. 26 shows the results of analyzing the effect of mutation burden on a prediction model in which different types of feature information are merged according to the present invention.

### DETAILED DESCRIPTION

Hereinafter, exemplary embodiments of a system and method of predicting drug indications and drug response using an artificial intelligence (AI) deep learning model based on the convergence of different types of feature information according to the present invention.

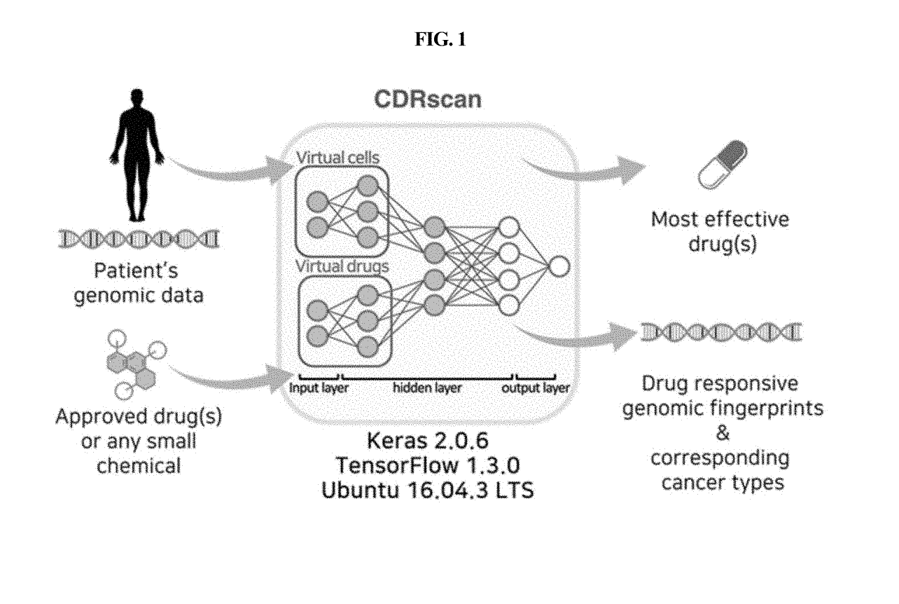

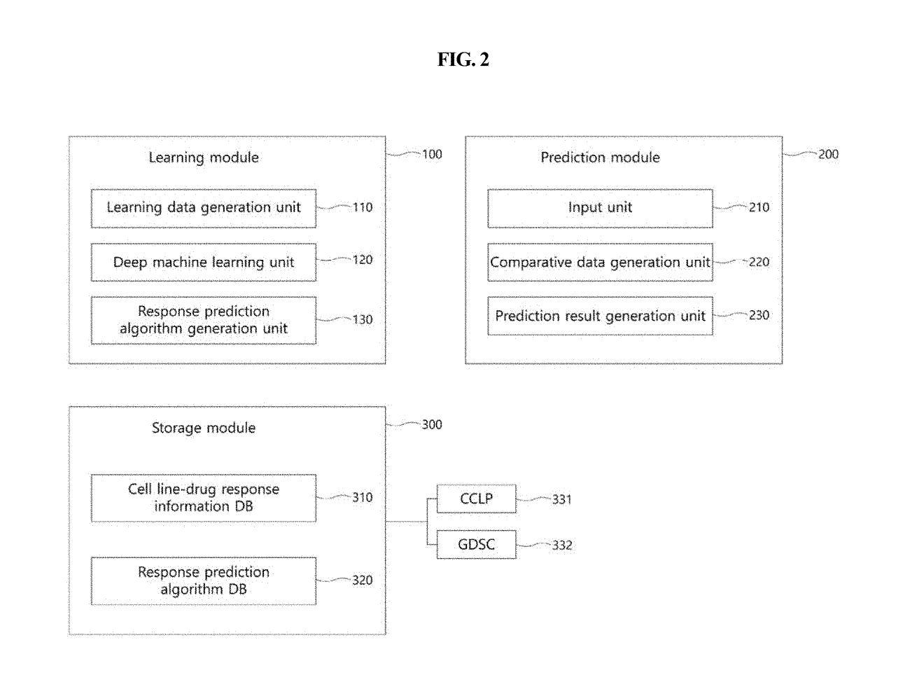

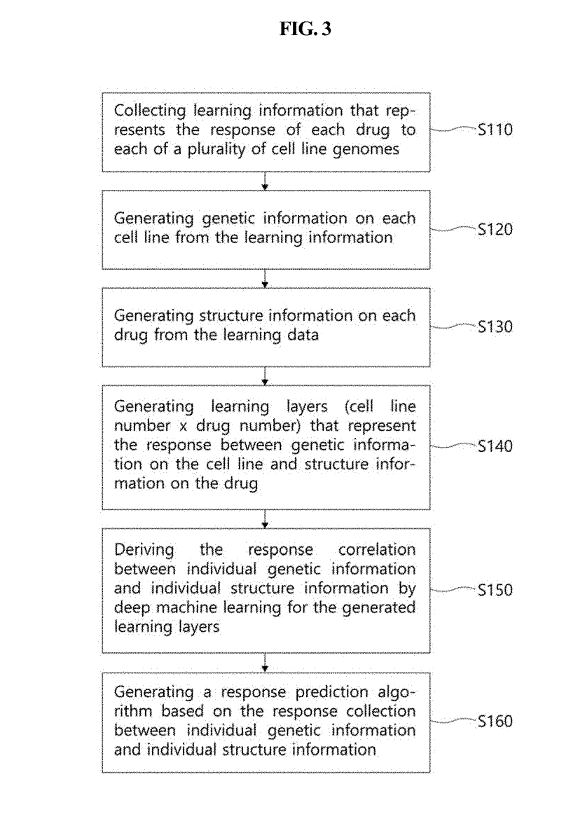

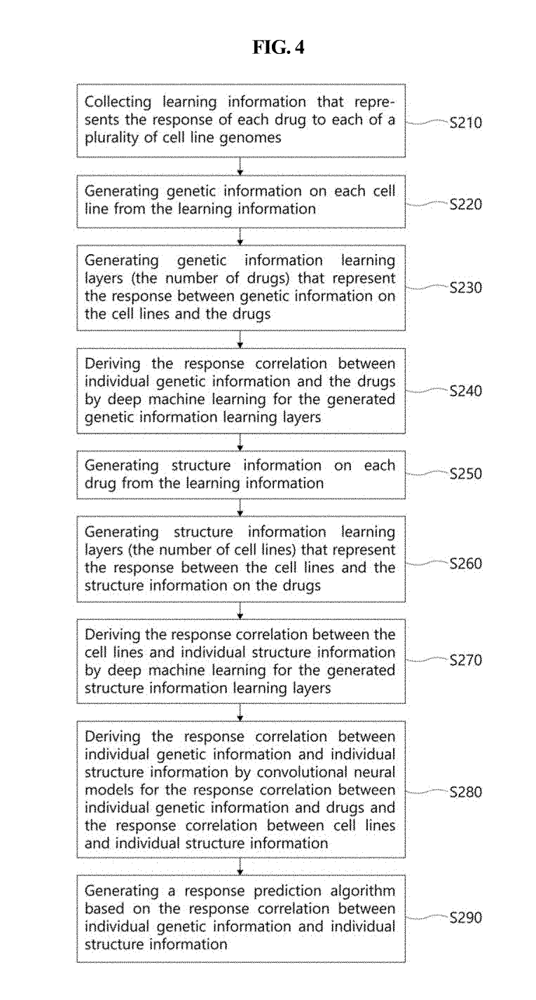

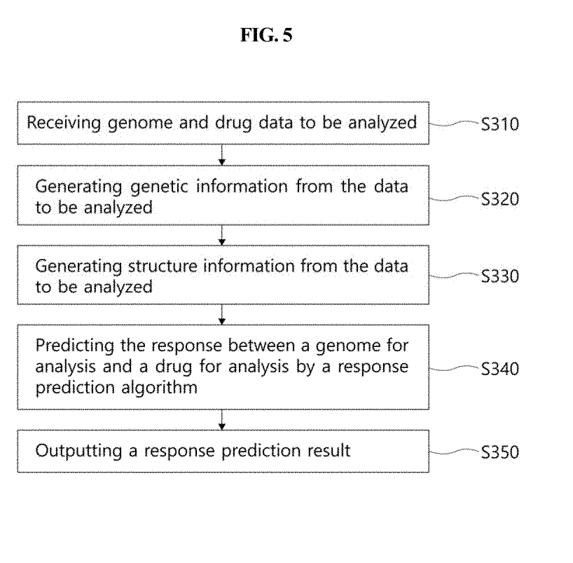

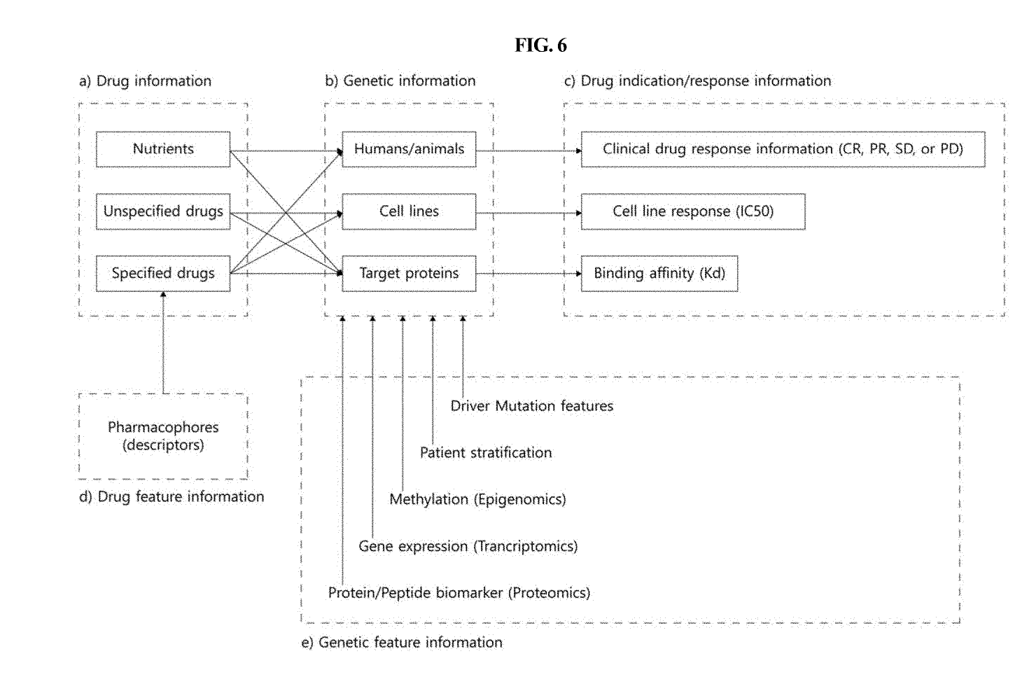

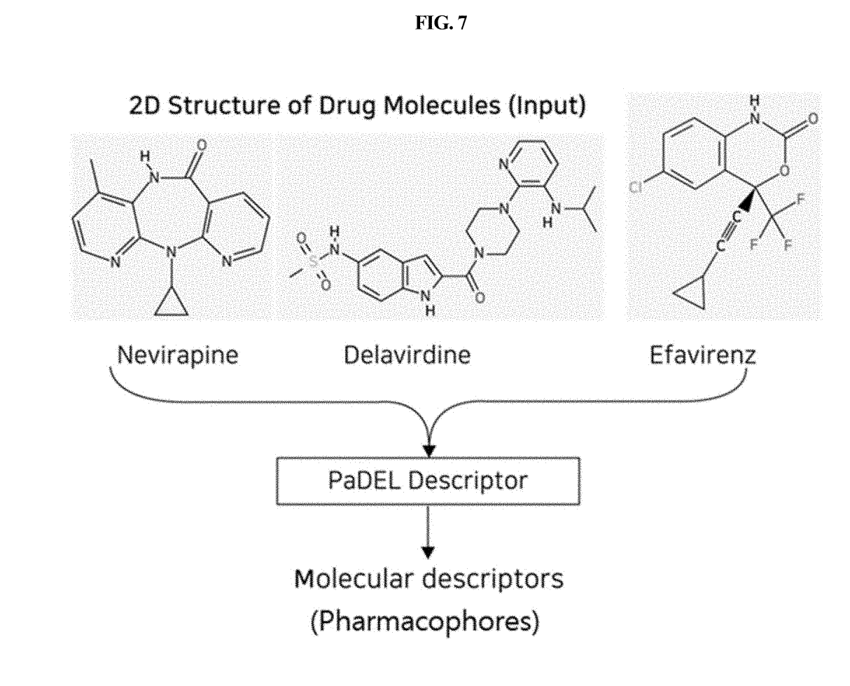

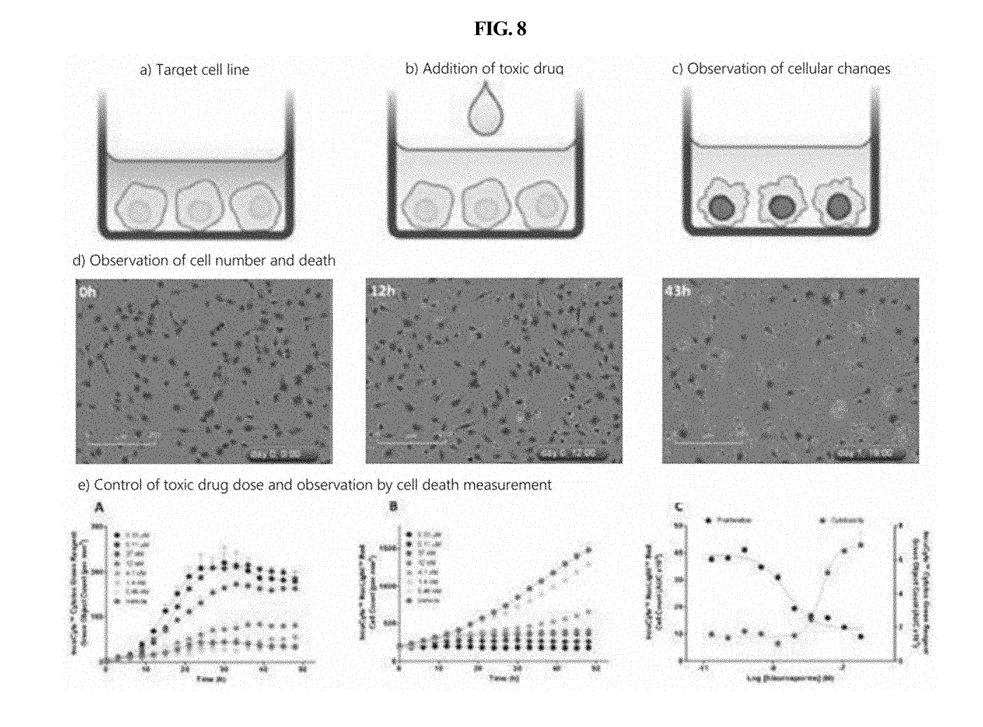

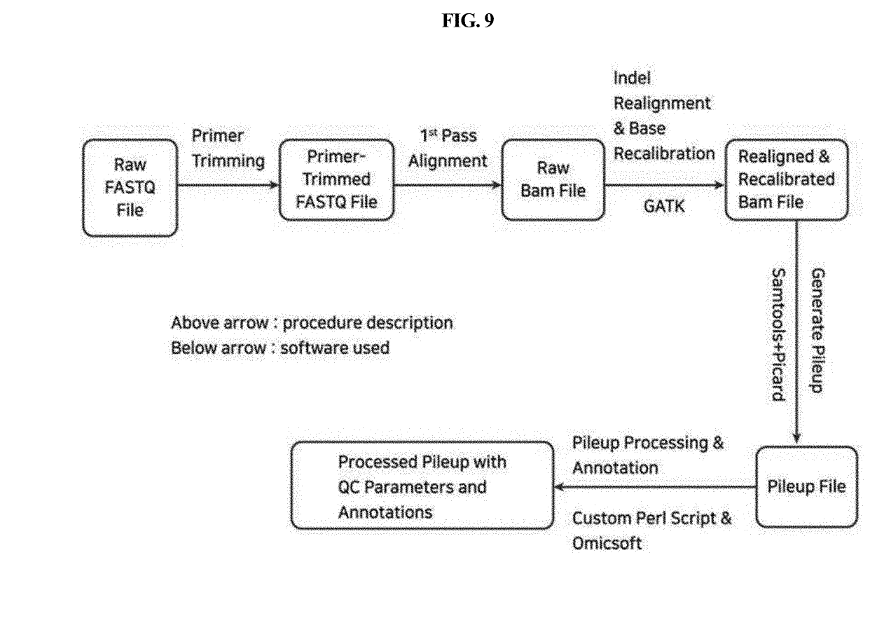

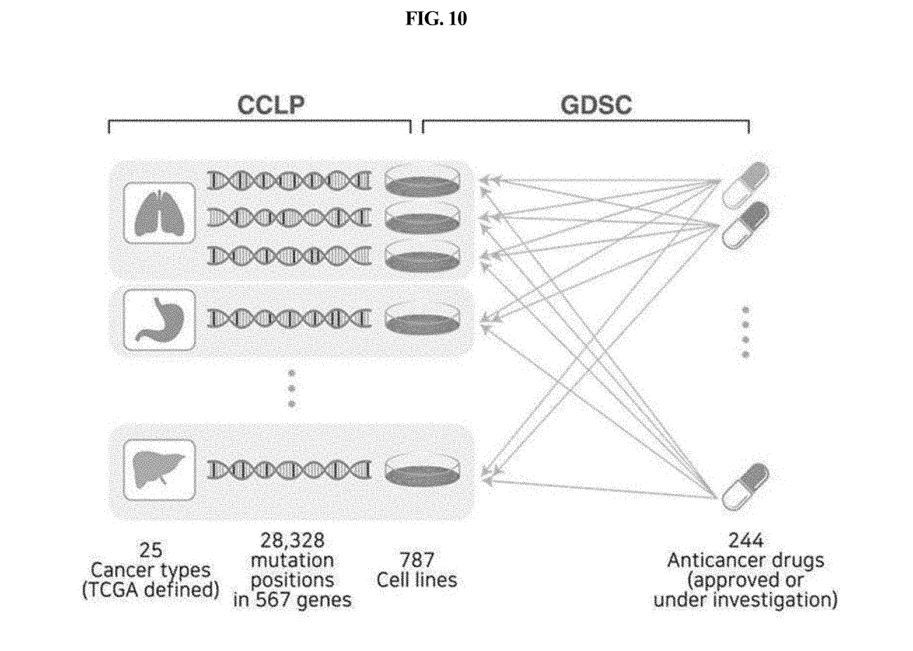

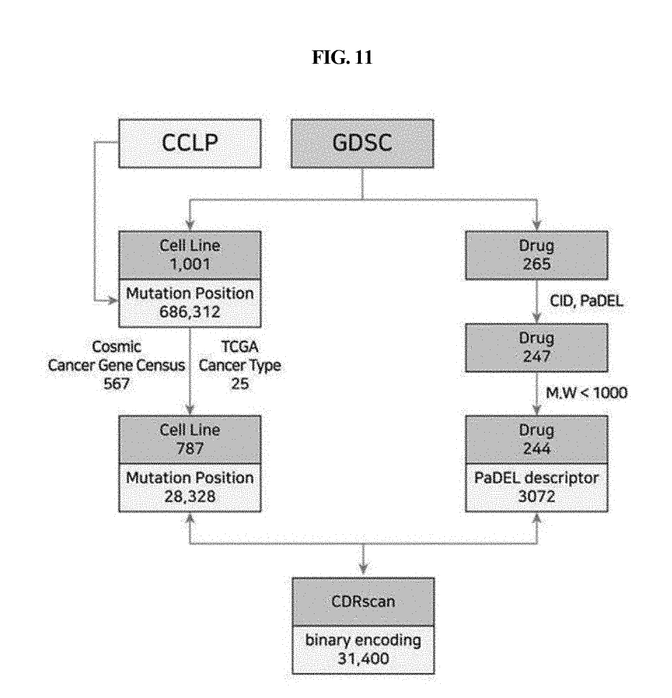

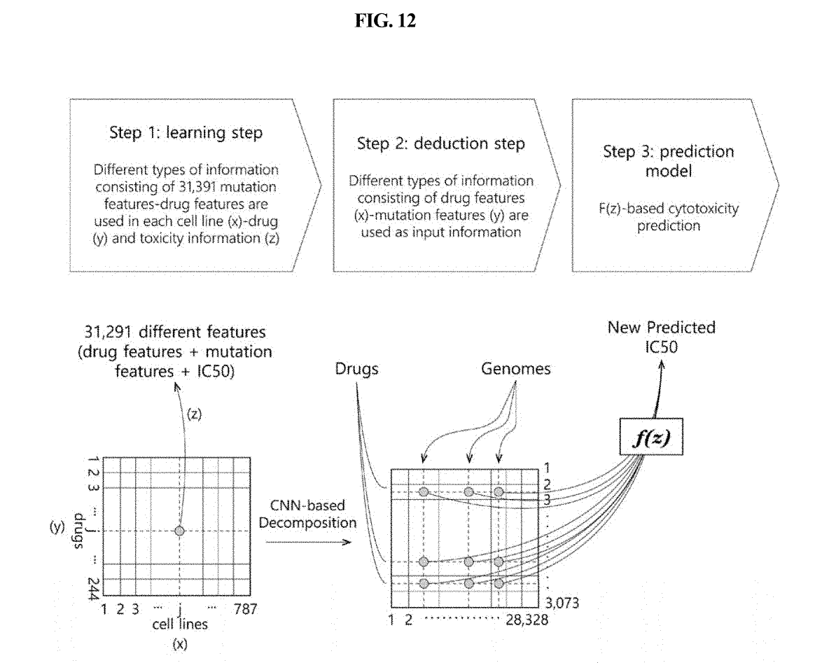

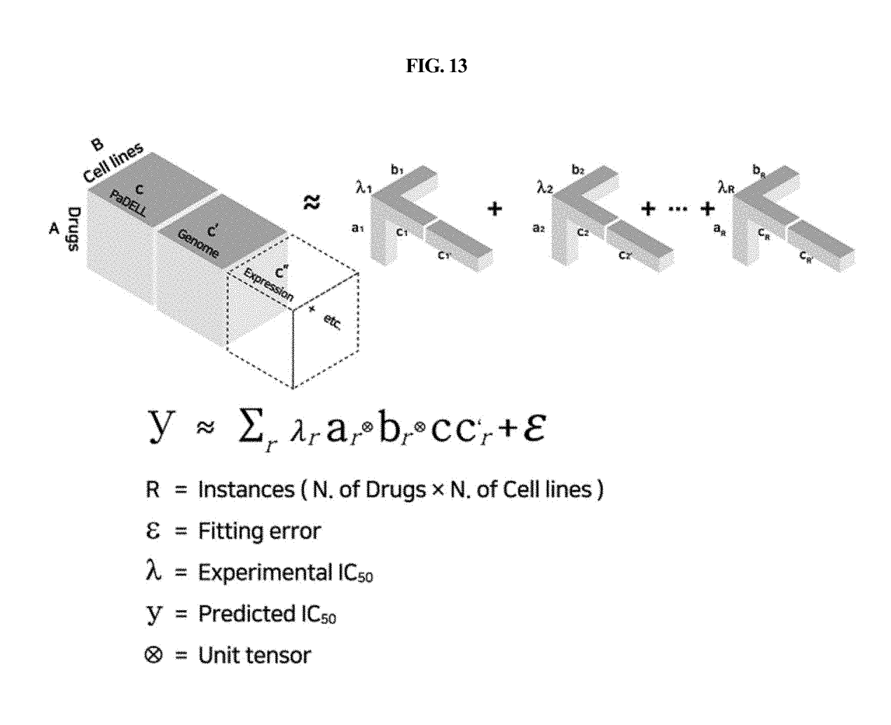

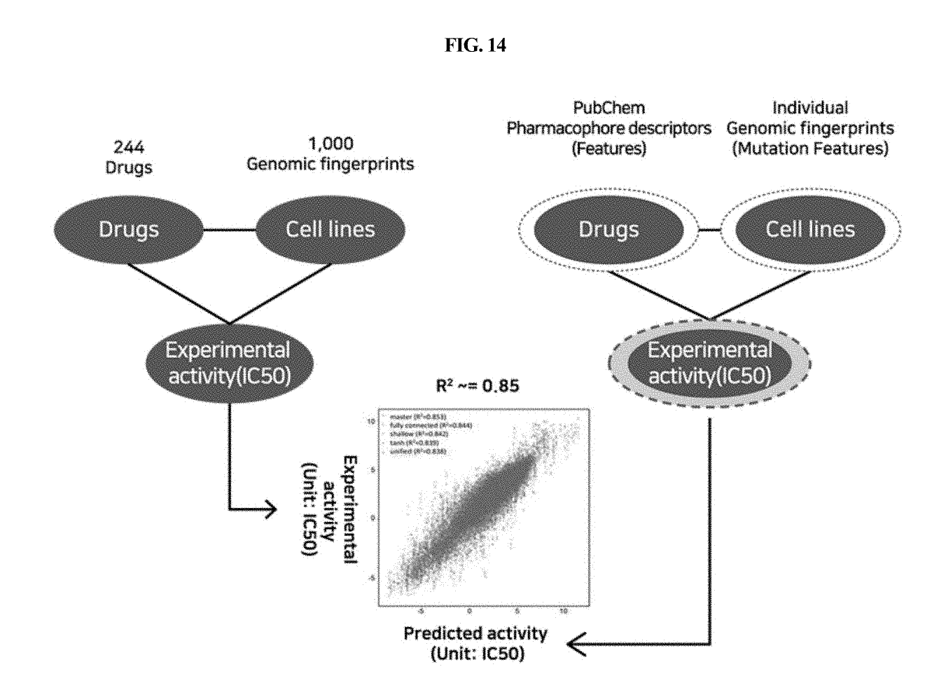

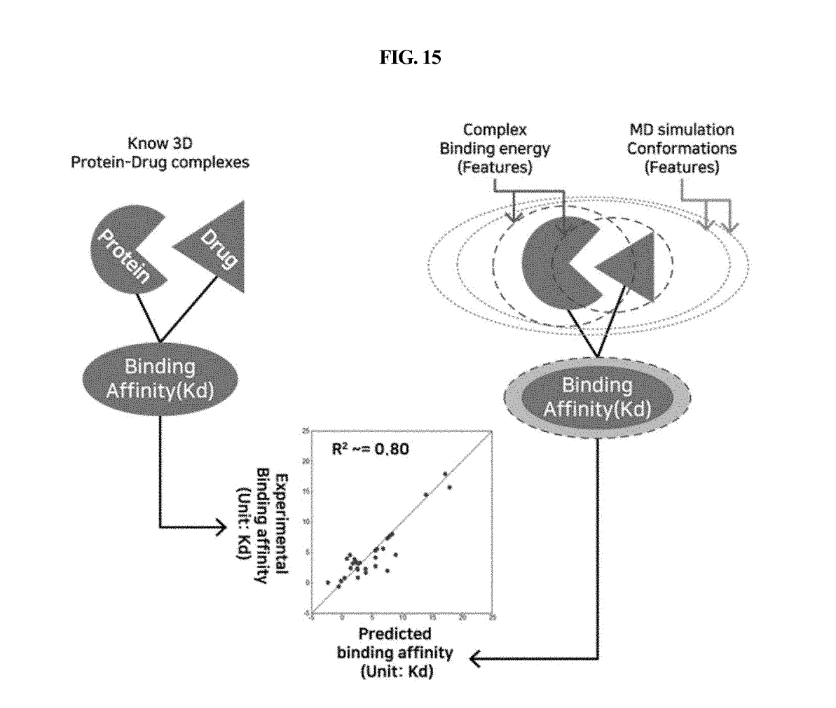

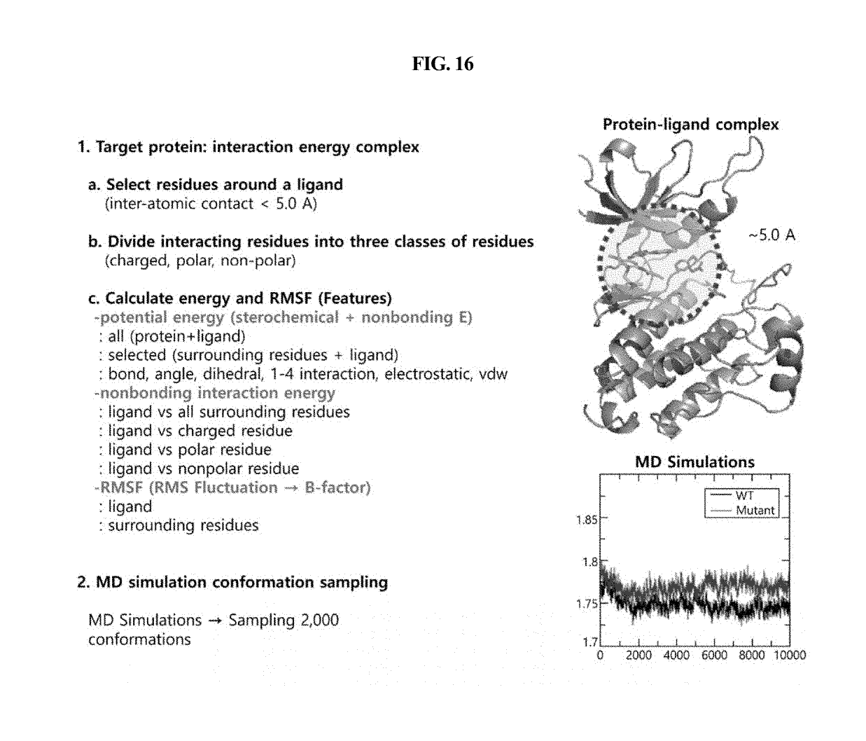

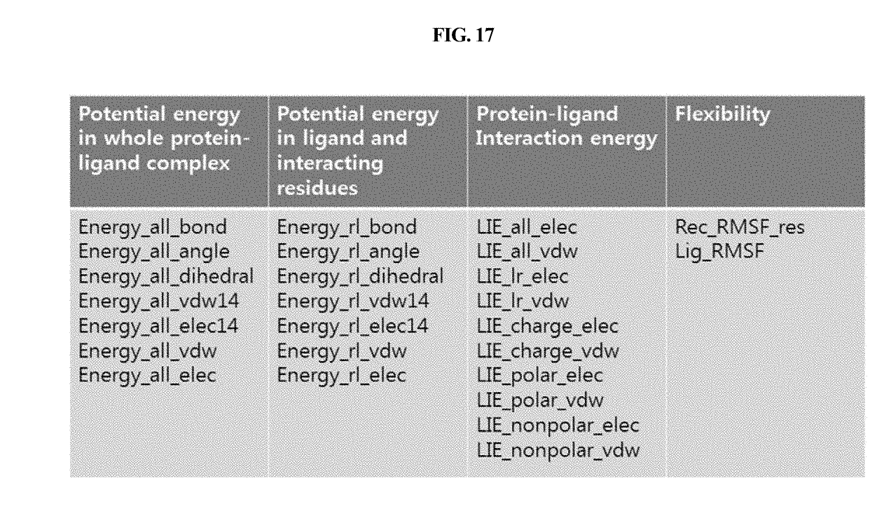

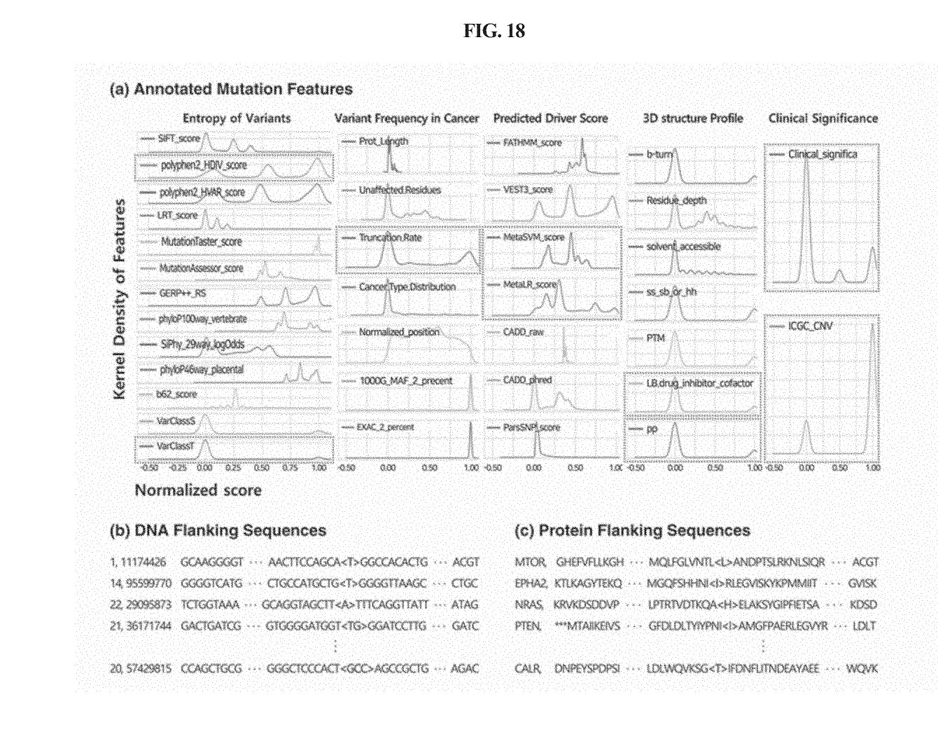

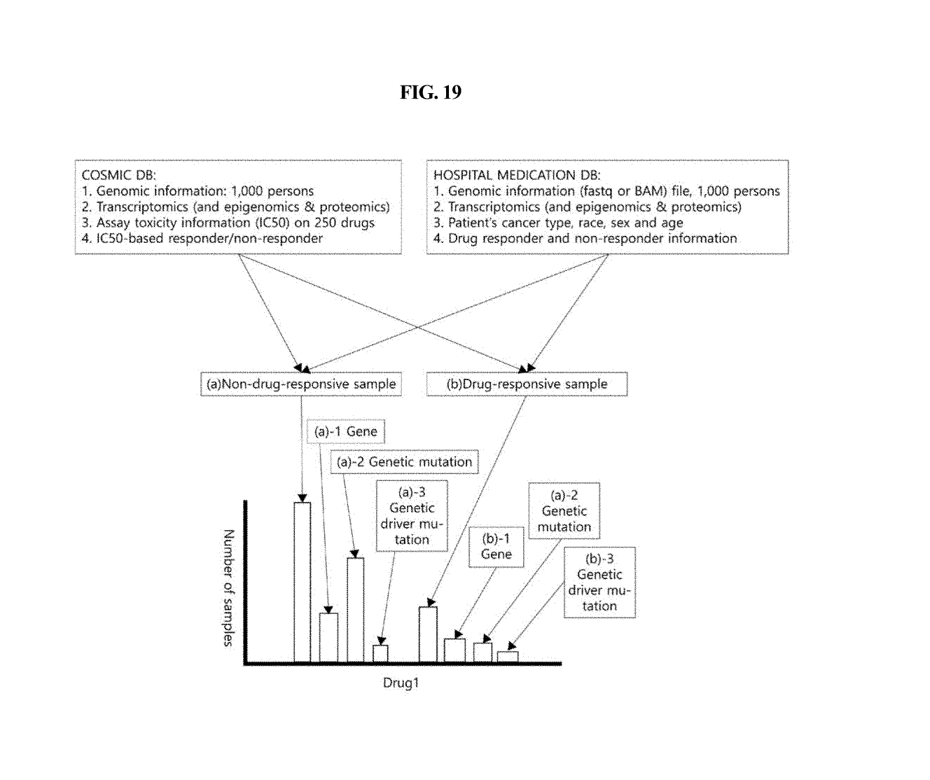

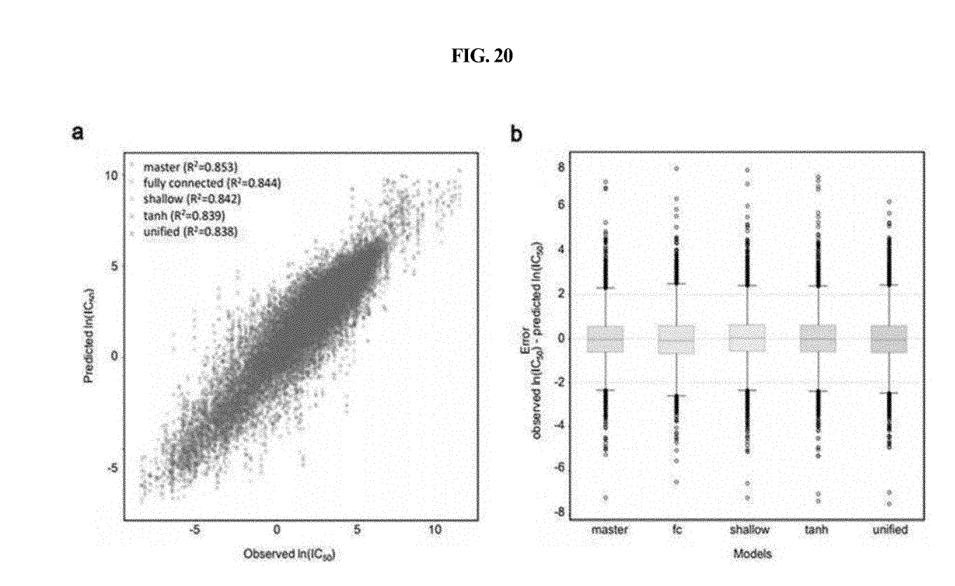

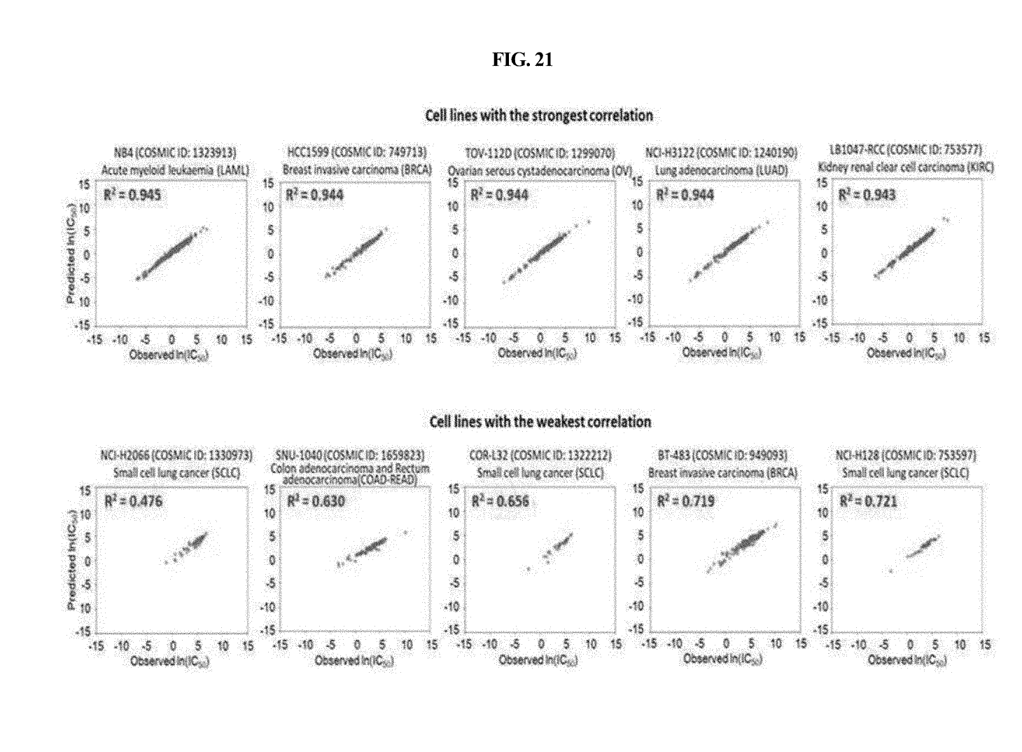

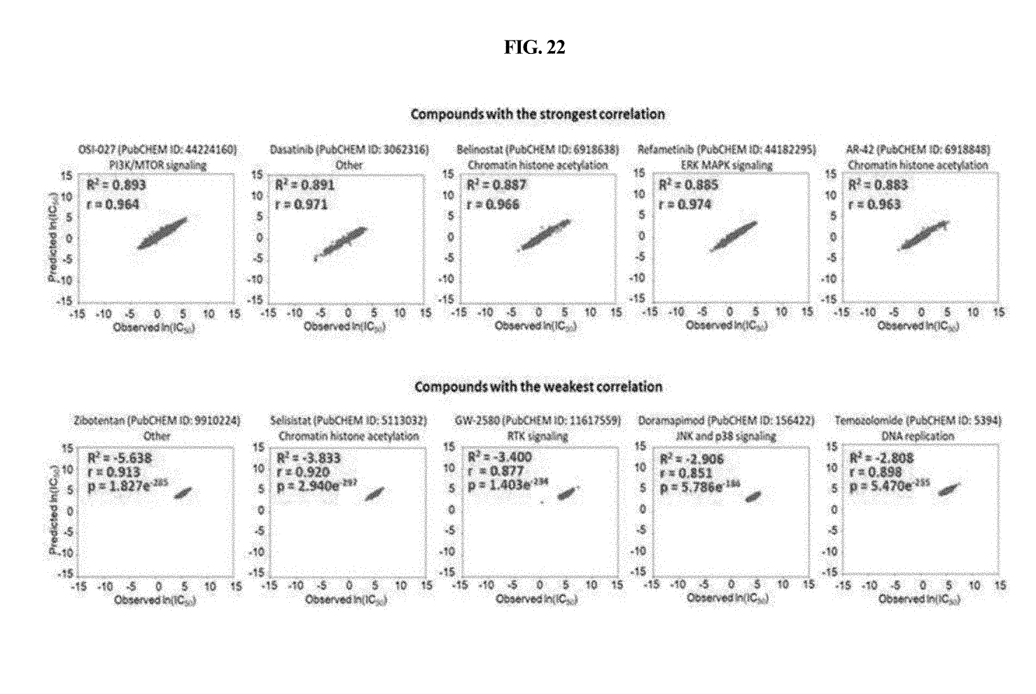

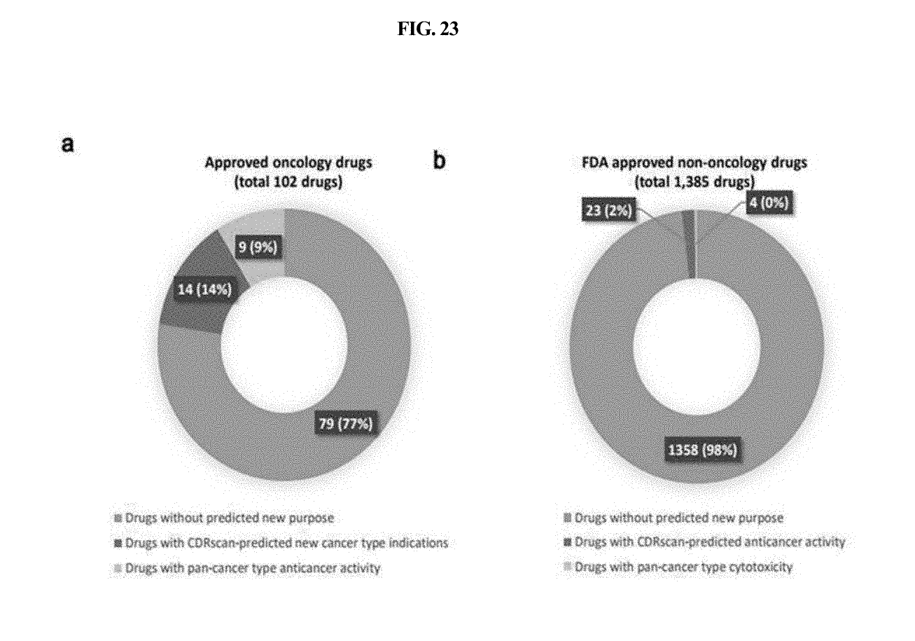

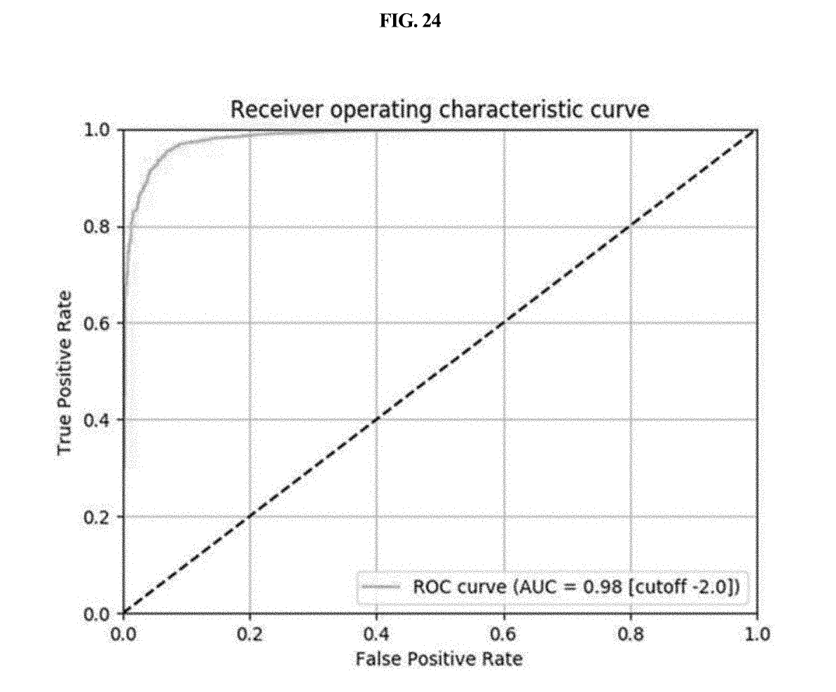

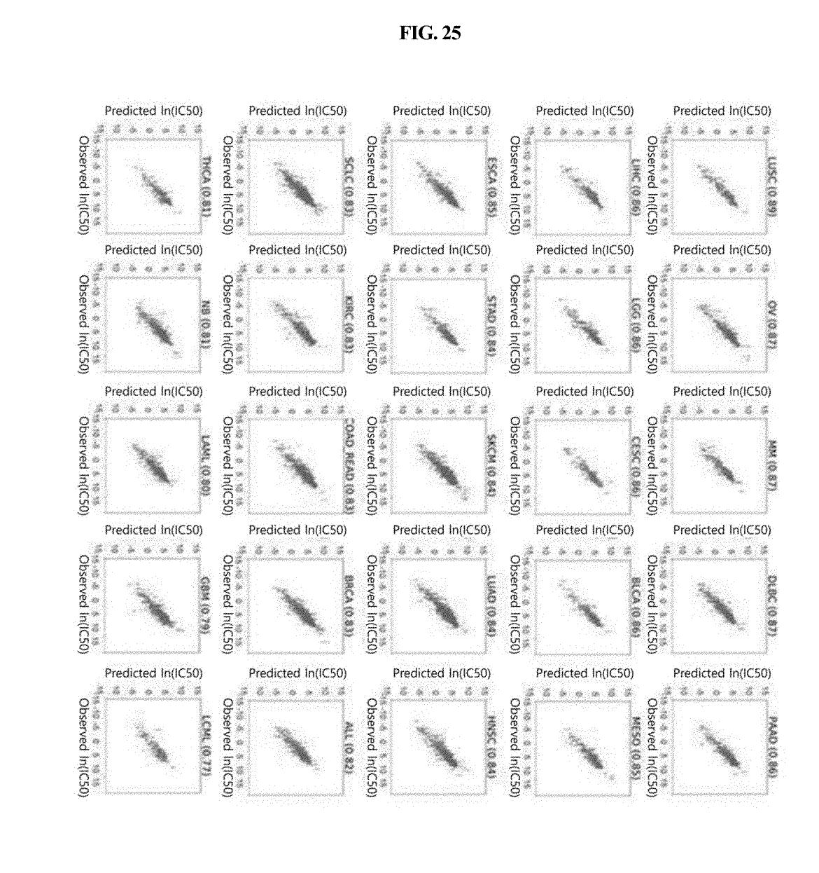

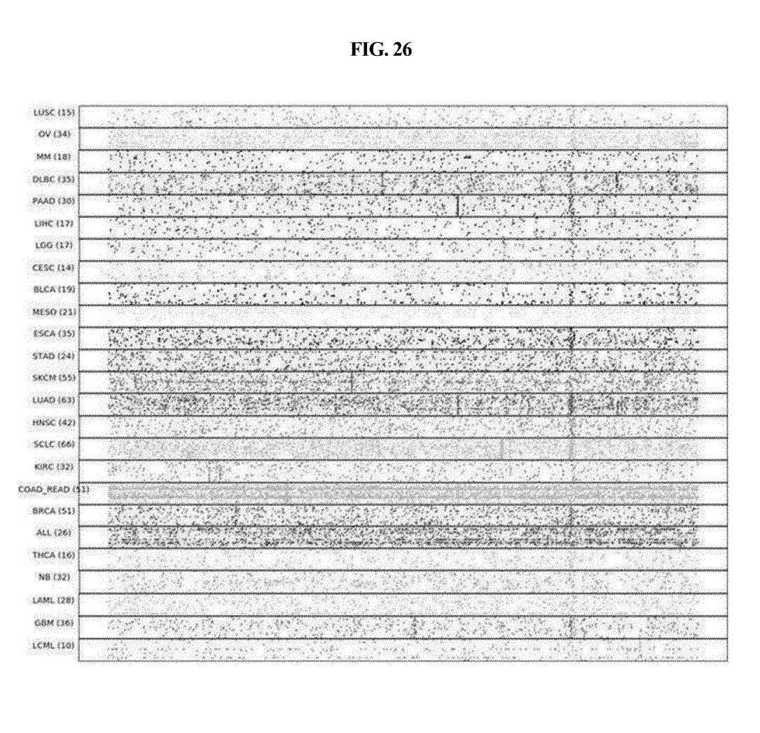

FIG. 1 illustrates one example of the deep machine learning structure of a CDRscan according to the present invention; FIG. 2 is a block diagram showing the configuration of a drug indication and drug response prediction system of the present invention, divided according to function; FIG. 3 is a flow chart showing one example of a deep machine learning method which embodies a drug indication and drug response prediction method of the present invention; FIG. 4 is a flow chart showing another example of a deep machine learning method which embodies a drug indication and drug response prediction method of the present invention; FIG. 5 is a flow chart showing one example of a drug response prediction method which embodies a drug indication and drug response prediction method of the present invention; FIG. 6 illustrates drug information, genetic information, and information on the responsiveness and features thereof for deep machine learning according to the present invention; FIG. 7 illustrates one example of a PeDEL pharmacophore descriptor according to the present invention; FIG. 8 illustrates one example of a process for generating IC50 data for a drug which is applied in the present invention; FIG. 9 illustrates one example of the configuration of a process for generating genetic information on a cell line according to the present invention; FIG. 10 illustrates a structure for generating data on the relationship between disease-related genome and drug toxicity, which is used in the present invention; FIG. 11 illustrates a process for generating data on the relationship between disease-related genome and drug toxicity according to the present invention; FIG. 12 illustrates an example of each step of a deep machine learning method according to the present invention; FIG. 13 illustrates one example of a merged structure of different types of information for deep machine learning according to the present invention; FIG. 14 shows cell line-based drug toxicity experiment data and drug response prediction results according to the present invention; FIG. 15 shows the results of predicting drug binding affinity based on a target protein according to the present invention and drug binding affinity by simulation; FIG. 16 illustrates simulations and drug interaction energy data sources for calculation of target protein-drug binding affinity according to the present invention; FIG. 17 illustrates drug interaction energy data sources for calculation of target protein-drug binding affinity according to the present invention; FIG. 18 illustrates mutation features, DNA flanking sequences and protein flanking sequences; FIG. 19 illustrates experiments which embody in vitro and in vivo drug indication and response prediction methods according to the present invention; FIG. 20 illustrates correlation (R²) values for the drug indication and drug response prediction results according to the present invention; FIG. 21 illustrates the results of obtaining correlation (R²) values for the drug indication and drug response prediction results for each cell line according to the present invention; FIG. 22 illustrates the results of obtaining correlation (R²) values for the drug indication and drug response prediction results for each drug according to the present invention; FIG. 23 shows the results of predicting new applications of conventional drugs according to the present invention; FIG. 24 shows the result of generating an ROC-curve for the accuracy of a prediction model in which different types of feature information are merged according to the present invention; FIG. 25 illustrates the results of obtaining R² values for individual cancer types by a prediction model in which different types of feature information are merged according to the present invention; FIG. 26 shows the results of analyzing the effect of mutation burden on a prediction model in which different types of feature information are merged according to the present invention.

The system for predicting drug indications and drug response according to the present invention will be hereinafter referred to as the CDRscan. In order to facilitate understanding of the present invention, the functional configuration and the method of performing the system according to the present invention will be described first, and then various embodiments and experimental examples according to the present invention will be described.

As shown in FIG. 1, the CDRscan according to the present invention is a machine learning system that predicts the drug (anticancer drug) response (IC50) of a disease of interest from mutation information (genomic signature) on a cell line with a particular disease (tumor).

The CDRscan is similar to a convolutional neural network (CNN) model, but determines the response of the drug of interest by calculating response (IC50) values predicted by different machine learning models (five) designed independently.

As the different machine learning models, various deep learning models may be used. These models can be largely classified into: 1) a method that performs machine learning using genetic information and structure information, which are to be finally analyzed, as learning elements; and 2) a method that comprises performing machine learning using genetic information and a drug, performing machine learning using a genome and structure information as learning elements, calculating the first learning relationship, and then performing second learning on such information.

Hereinafter, the configuration and method of the present invention for embodying and performing this CDRscan will be described with reference to FIGS. 2 to 5.

First, as shown in FIG. 2, a specific example of the system for predicting drug indications and drug response according to the present invention comprises a learning module 100, a prediction module 200 and a storage module 300.

Here, the learning module 100 is configured to learn response correlation between structure information on drugs and genetic information on genomes by deep machine learning from collected learning information.

Here, the learning information is information on the response of cell lines to drugs and is collected from the Cancer Cell Line Encyclopedia (CCLE) or Genomics of Drug Sensitivity in Cancer (GDSC) databases.

Meanwhile, to perform this function, the learning module 100 comprises a learning data generation unit 110, a deep machine learning unit 120 and a response prediction algorithm generation unit 130.

Here, the learning data generation unit 110 is configured to generate learning data for deep machine learning from the collected learning information; the deep machine learning unit 120 is configured to perform deep machine learning of a number of learning data generated from the learning data generation unit; and the response prediction algorithm generation unit 130 is configured to generate a response prediction algorithm, which predicts the response of the genome to the drug, from the results learned by the deep machine learning unit 120.

At this time, the genetic information and the structure information can be variously set according to information on deep learning units. That is, each of the genetic information and the structure information may be set as subunit information of each of the genome and the drug (compound) or as various information contained therein.

Although the present invention discloses an example in which mutation information on the genome and feature information on the mutations are set as the genetic information, it is also possible to set nucleotide sequence information as the genetic information if hardware is supported.

Likewise, although the present invention discloses an example in which descriptor information is set as the drug structure information, it is also possible to set the entire functional group of the drug as the drug structure information.

Namely, in the present invention, the accuracy of response prediction results increases the number of elements common between subjects on which machine learning was performed and subjects whose information was input for analysis increases. Thus, if the units of the genetic information and the analysis information are set in detail, the accuracy of prediction of response to an unknown compound can be increased.

In a specific embodiment of the present invention, the case in which mutation information is set as the genetic information will be explained with the case in which feature information is set as the genetic information. In the case in which mutation information is set as the genetic information and deep machine learning is performed, the accuracy of analysis increases as the number of elements common between cell line variations contained in learning information and genomic mutations input for analysis increases.

On the other hand, in the case in which feature information is set as the genetic information and deep machine learning is performed, response can be accurately predicted due to similar features of mutations, even though the number of elements common between cell line variations contained in learning information and genomic mutations input for analysis is small.

Thus, in this case, the response of genomes of species having different mutation features to drugs can be predicted.

As such, the structure information may be descriptor information on the drug.

In addition, the genetic information may be mutation information on the genome or feature information on the mutations contained in the genome.

When the genetic information is mutation information, the learning data are a number of information that indicate the responsiveness of a group of mutation information on the cell line to a group of descriptor information on the cell line.

On the other hand, the genetic information is feature information on mutations, the learning data are a number of information that represents the response between a group of feature information on mutations contained in the cell line and a group of descriptor information on the mutations contained in the cell line.

The feature information is genomic fingerprints on the mutations, and may comprise any one or more of mutability or entropy of variants, variant frequency in cancer, driver mutation score, 3D structure mutation environment, clinical significance mutation, drug response stratification attributable to genetic interaction, epigenomics, transcriptomics, and proteomics.

Meanwhile, deep machine learning unit 120 learns response correlation between each drug structure information and each genetic information on the cell line by deep machine learning on the learning data.

Here, the deep machine learning may be performed by various deep learning techniques. It may typically be performed by TensorFlow machine learning, a Google open source. More specifically, it may be performed by a Convolutional Neural Network (CNN) model.

Hereinafter, a specific example of the deep machine learning method by the learning module will be described with reference to FIGS. 3 and 4.

The deep machine learning method according to the present invention is divided into two methods. First, a method of performing machine learning using genetic information and structure information, which are to be finally analyzed, as learning elements, will be described with reference to FIG. 3.

As described in FIG. 3, the first method of the deep machine learning method according to the present invention starts with the learning data generation unit collecting learning information that indicates the response of each cell line genome to each drug (S110).

Here, the learning information refers to experimental result data on the response of various cell lines to various drugs.

Thereafter, the learning data generation unit generates genetic information on the genomes from the learning information (S120).

Here, the genetic information may be mutation information or feature information on mutations.

Furthermore, the learning data generation unit generates structure information on the drugs from the learning information (S130).

Here, the structure information may be descriptor information on the drugs.

Afterwards, the learning data generation unit generates learning layers, which represent the response between a group of genetic information on the genomes and a group of structure information on the drugs, from the learning information.

Here, the learning layers are merged data for application to the CNN model, and specific examples thereof are shown in FIGS. 12 and 13.

At this time, the learning layers are theoretically generated by the number of cell lines×the number of drugs, from the learning information.

Next, the deep machine learning unit derives the response correlation between individual structure information and individual genetic information by deep machine learning on the learning layers.

Here, the results of response to the drugs and the criteria of prediction may be judged on the basis of the inhibition index IC50.

The IC50 means the concentration of the drug required to kill 50% of the cells of the cell line. The lower the IC50 value, the higher the reactivity of the drug.

Next, the response prediction algorithm generation unit generates an algorithm that predicts the response between genetic information on the genome and structure information on the drug, through the response correlation between the genetic information and the structure information, learned by the deep machine learning unit (S160).

At this time, the deep machine learning unit 120 may be configured to perform the deep machine learning by a plurality of methods (models), and then calculate final prediction values from the mean of prediction values.

As shown in FIG. 4, the second method of deep machine learning according to the present invention starts with the learning data generation unit collecting learning information that indicates the response of each cell line genome to each drug (S210).

Furthermore, the learning data generation unit generates genetic information on the genomes from the learning information (S220).

Also in this case, the genetic information may be mutation information or feature information on mutations.

Next, the learning data generation unit 100 generates genetic information learning layers that indicate the response of a group of the genetic information on each genome (S230) to drugs.

Furthermore, the deep machine learning unit 120 derives the correlation between drug response and each genetic information by deep machine learning on the genetic information learning layers (S240).

Next, the learning data generation unit 110 generates structure information on the drugs from the learning information (S250).

Thereafter, it generates structure information learning information that represents the response between drug structure information and each genome (S260), and it derives the response correlation between each structure information and each genome by deep machine learning for the structure information learning layers (S270).

In addition, the deep machine learning unit 120 derives the response correlation between individual structure information and individual genetic information through the correlation between drug response and each genetic information, determined in step 240, and the correlation of the response of each structure information to each genome, determined in step 270.

When the number of genetic information on genomes and the number of structure information on drugs are large, this second method of deep machine learning not only can disperse the deep machine learning process to separate the process into two, but also can improve the accuracy of the correlation.

Meanwhile, the prediction module 200 is configured to receive analysis information and to output the result of predicting the response of the genome to the drug on the basis of the analysis information. To this end, the prediction module comprises an input unit 210, a comparative data generation unit 220, and a prediction result generation unit 230.

At this time, the input unit 210 is configured to be input with the information to be analyzed, and the information to be input refers to information containing the genome and drug data to be analyzed.

Furthermore, the comparative data generation unit 220 is configured to generate comparative data corresponding to genetic information and structure information, which are used in deep machine learning, from the genome and drug data contained in the information to be analyzed, respectively.

Namely, when the deep machine learning is performed using mutation information and descriptor information, then the comparative data generation unit 220 generates mutation information on the genomes from the analysis information, and generates descriptor information from the analysis information.

Of course, when the deep machine learning is performed using feature information and descriptor information, then the comparative data generation unit generates feature information on the genomic mutations from the analysis information, and generates descriptor information from the analysis information.

The prediction result generation unit 230 is configured to output the result of prediction of the response of the genomes contained in the analysis information to the drugs by the response prediction algorithm derived by the response prediction algorithm generation unit 130.

Hereinafter, a specific example of the method of predicting drug response by the prediction module will be described with reference to FIG. 5.

As shown in FIG. 5, the response prediction method according to the present invention starts with the input unit 210 receiving the analysis information containing the genome and drug data to be analyzed (S310).

Then, the comparative data generation unit 220 generates genetic information on the genomes from the analysis information (S320), and generates structure information on the drugs from the analysis information (S330).

At this time, as described above, the structure information and genetic information to be analyzed correspond to structure information and genetic information, respectively, applied to the deep machine learning, and may be descriptor information on drugs and mutation information on the genomes or feature information on the mutations contained in the genomes.

In addition, it outputs the result of prediction of the response of the genomes contained in the analysis information to the drugs, based on the response correlation between the structure information and the genetic information by the response prediction algorithm, and outputs the generated result (S340 and S350).

Meanwhile, the storage module 300 is configured to store the response prediction algorithm learned by the learning module, and may comprise a response prediction algorithm DB 320 and may further comprise a cell line-drug response DB 310 for storing collected learning data.

Hereinafter, embodiments of the system and method for predicting drug indications and drug response according to the present invention will be described with reference to the accompanying drawings.

As described above, in the deep machine learning of the CDRscan according to the present invention, the first step of the example comprising two consecutive steps extracts 28,328 and 3,072 features from the genomic sequence data and the chemical characteristics of anticancer drugs, respectively.

These features can be regarded as the genomic mutational fingerprints of cancer cell lines and the molecular fingerprints of drugs.

Then, each set of fingerprints are individually convoluted using a Convolutional Neural Network (CNN) model, thereby generating virtual tumor cells and virtual drugs.

Next, ‘virtual docking’ which is drug response is performed, and predicted IC₅₀ values across a plurality of anticancer drugs (244 drugs) for each virtual cell line are examined.

This CDRscan can generally be applied to two fields.

First, the CDRscan can be used in clinical practice to predict the most effective anticancer drug for a specific genomic signature of a cancer patient.

In addition, the CDRscan may be used to examine the sensitivity of somatic mutations to a particular drug or a small compound.

Furthermore, cancer types can be predicted according to a genomic signature expected to be sensitive to a particular compound.

To realize this CDRscan, the CDRscan uses software and hardware as described below.

Namely, in the present invention, the CDRscan uses software of TensorFlow 1.3.0, Keras 2.0.6 and Ubuntu 16.04.3 LTS in combination in order to implement CNN (convolution neural network).

In addition, the CDRscan uses a workstation equipped with NVidia GTX 1080Ti as hardware in order to perform the design, training and verification of the above-described system on the basis of GPU.

Meanwhile, in the CDRscan model, two different sources of input are used, which represent the genomic sequence variations of individual cancer cell lines and the chemical properties of anticancer drugs, respectively.

Here, the genomic fingerprints of cancer cell lines are expressed as a string of 28,328 binary codes, each representing a somatic mutation status.

At this time, the presence of a somatic mutation was encoded as 1 and absence as 0. The molecular fingerprints of 244 GDSC drugs are encoded using 3,072 binary descriptors.

Meanwhile, a line notation of simplified molecular-input line entry system (SMILES) is initially generated from structure information obtained from PubChem (Kim S, Thiessen P A et al) for each drug.

Next, a PaDEL-descriptor (v2.2.1) is used to extract descriptors of three classes of fingerprints: fingerprinter, extended fingerprinter, and graph only fingerprinter.

Hereinafter, the principle of the deep machine learning according to the present invention will be described in detail with reference to FIGS. 12 and 8-2.

As shown in FIGS. 12 and 8-2, in the CDRscan according to the present invention, different types of information are merged and subjected to deep learning. The different types of information may be mutation and feature information on cell lines and descriptor information or phenotype information on drugs.

Namely, these different types of information are arranged according to cell lines and drugs, and these merged data are learned by deep machine learning.

At this time, the algorithm of the machine learning may be defined by the equation shown in FIG. 13.

Meanwhile, in the present invention, drug descriptors are used in the deep machine learning and prediction processes. As shown in FIG. 7, the use of drug descriptors increases the efficiency of learning and analysis compared to when polymer compounds for drugs are used intact.

Meanwhile, the NGS data of cell lines that are used in the present invention are generated through a pipeline as shown in FIG. 9.

The genomic data generation pipeline shown in FIG. 9 has already verified its accuracy and reliability, and thus the detailed description thereof is omitted herein.

Meanwhile, as described above, learning data for the deep learning according to the present invention are extracted from two major databases (CCLP and GDSC) as shown in FIG. 10.

These provide comprehensive public databases for genomic profiles of human cancer cell lines and drug sensitivity assays.

The CCLP includes somatic mutations of 1,000 or more cancer cell lines from broad cancer types, and the GDSC includes drug sensitivity analysis results for 1,000 or more CCLP cancer cell lines and 265 anticancer drugs.

The entire datasets from these databases contain 686,312 mutation positions from 1,001 cell lines and 265 drugs.

Meanwhile, in the present invention, these data are filtered according to the following criteria and used.

First, gene mutations contained in Cancer Gene Census are used, and the mutations are judged from a catalogue of 567 genes associated with cancer pathology.

Second, only cancer types which are shown at least 21 different cell lines are used.

Of 31 cancer types consisting of 1,001 cancer lines, 25 cancer types with a total of 787 cell lines are contained in datasets.

Meanwhile, particular cancer types may be excluded. For example, particular cancer types are expressed as a relatively small number of cell lines, these cancer types can be excluded from assessment.

The CCLP contains various types of molecular profile data, including whole exome sequencing data of 1,001 human cancer cell lines commonly used in cancer research.

In one example of the present invention, sequence variation information at 28,328 positions from 567 genes in the COSMIC Cancer Gene Census was selected.

The GDSC provides IC₅₀ values from drug sensitivity assays for over 200,000 drug-cancer cell line pairs.

At this time, the IC₅₀ is used as a criterion for determining activity for drug response, and 50% is usually used as a criterion, but data set by other criteria may also be applied.

In GDSC, the identical set of 1,001 cell lines genomically characterized by CCLP was used, and 265 anticancer therapeutics from various sources, ranging from FDA-approved drugs to those under investigation, were included in the assays.

Meanwhile, in the present invention, a line notation of simplified molecular-input line entry system (SMILES) is used to extract the structural and chemical features of each drug.

However among 265 drugs, 18 drugs were registered in SMILES, and three drugs had a molecular weight exceeding 1,000 g/mol. These 21 drugs were removed from the dataset.

At this time, in GDSC, some identical chemicals can be counted as two discrete entities.

There were 9 such pairs, but since the IC₅₀ values were different across all pairs, the 9 pairs could be considered as 18 distinctive drugs in order to perform learning.

Namely, in one example of the present invention, the final dataset had 244 drugs representing 229 individual small chemicals. A total of 152,594 instances were in the final matrix of cell lines and drugs and employed in the deep machine learning.

In one example of the deep machine learning according to the present invention, prediction of 25 particular cancers, about 1,000 cancer cell lines and the activity of about 250 anticancer drugs can be performed through the following procedures as shown in FIG. 8:

1) All available data of CCLP and GDSC databases from COSMIC data are analyzed/extracted to obtain data about a total of 200,000 cancer cell cases vs. cytotoxic activity (=potential as cancer therapeutics) of about 250 drugs.

2) Then, for a total of 200,000 clinical/experimental data, deep machine learning is performed by the above-described CDRscan using TensorFlow.

3) In addition, to verify the performance of the CDRscan, the performance is accessed by 5-fold-cross validation for all the data of step 1).

In one example of the present invention, accuracy corresponding to a Pearson correlation coefficient of 0.9 or higher was confirmed in a total of 25 cancer cell types.

As described above, the present invention is based on two distinct types of data which are learning data for machine learning.

One includes the genomic features of cell lines expressed as 28,328 descriptors, and the other includes chemical properties with 3,072 PaDEL descriptors. Thus, the input features of the entire instance are represented by a total of 31,400 descriptors.

Of the total of 152,594 instances spanning 25 cancer types, 144,953 instances (i.e., compilation of randomly selected 95% of instances for each cancer type) were selected to train all five models of CDRscan.

The remaining 7,641 instances (corresponding to 5% of the total instances) were set aside for evaluation of the accuracy of the models.

Thus, in the present invention, the reliability of the machine learning can be confirmed objectively.

Hereinafter, the deep machine learning method using the CDRscan will be described in detail with reference to FIG. 8.

As shown in FIG. 8, the deep machine learning using the CDRscan according to the present invention comprises a genomic CNN procedure, a PaDELL CNN procedure and a dual CNN procedure.

Here, the genomic CNN procedure refers to a process that classifies and sorts learning data according to a plurality of cell lines and a plurality of drugs and performs convolution-based learning for all genomic variations on the basis of response (IC₅₀).

The PaDELL CNN procedure refers to a process that classifies and assigns learning data according to a plurality of cell lines and a plurality of drugs and performs convolution-based learning for PeDELL descriptors on the basis of response (IC₅₀)—

The Dual CNN procedure refers to a process that performs a convolution-based learning in a state in which parameters for the genomic variations and PeDELL descriptor generated from the genomic CNN procedure and the PaDELL CNN procedure are merged.

Through these learning procedures, learning is performed in step 1 as shown in FIG. 12. In step 2, new genomic mutation features and pharmacophore descriptors are input, and then the response (IC50) of the genomic mutation features to the input pharmacophore descriptors can be predicted as shown in step 3.

Meanwhile, in drug response of cell lines as shown in FIG. 14, drug response in prospective/retrospective drug response clinical research as shown in FIG. 19, or target protein dissociation as shown in FIG. 15, the results of verifying the accuracy of the results of predicting drug dissociation using 2,000 simulation conformations and 26 interaction energies as shown in FIGS. 8-5 and 17 indicated that the R² value summarized in FIG. 15 was 0.80, indicating that the accuracy was very high.

Namely, as shown in FIG. 14, when compared with the actual in vitro experimental result value, the R² value was 0.85, and when compared with 3D simulation results for drug-protein binding (dissociation constant), the R² value was 0.8, and when an experiment was performed with the data of known drug information DB, the R² value was 0.85. Thus, it is considered that an in vivo method will show the same accuracy as the in vitro method shown in FIG. 19. In vivo clinical studies based on the present invention can be performed prospectively or retrospectively.

The above-described very high R² value compared to the R² value in conventional analysis methods (R² value: 0.6 to 0.7), indicates that the present invention shows a very high accuracy of prediction.

The IC₅₀ values predicted and observed for all the five models of the CDRscan according to the present invention show a strong correlation as shown in FIG. 20.

In the example shown in FIG. 20, the mean coefficient of determination (R²) value for the five models is 0.838 to 0.853, which is significantly higher than that of a conventional prediction model (Menden et al., 2013).

In all the five models, the mean error of predicted IC⁵⁰ value (i.e., predicted IC₅₀ minus observed IC₅₀) approaches 0, confirming that the prediction is accurate in most instances.

FIGS. 21 and 22 show correlations between predicted values and observed values for cell lines and drugs. Specifically, FIG. 21 shows examples of obtaining correlation (R²) values for the results of prediction of drug indications and drug response from a viewpoint of cell lines, and FIG. 22 shows examples of obtaining correlation (R²) values for the results of prediction of drug indications and drug response from a viewpoint of drugs.

Meanwhile, as shown in FIG. 23, the CDRscan according to the present invention may also be used to expand the application of drugs.

Namely, using the CDRscan according to the present invention, the sensitivities of 787 cell lines to all drugs (a total of 1,487 compounds) approved by the FDA were predicted. As a result, as shown in FIG. 23, chemical descriptors for 1,487 FDA-approved compounds were extracted, and the CDRscan generated a table of IC₅₀ values predicted for 787 cancer cell lines.

Among 1,487 drugs, 102 drugs were included in a GDSC anticancer drug panel.

The CDRscan analysis predicted applicability to additional cancer types in addition to original indications for 23 of the FDA-approved anticancer drugs.

Nine of these drugs showed an ln (IC50) of less than −2.0 in several cancer types and suggested non-specific cytotoxicity.

Fourteen drugs showed selectivity for only some of the cancer types.

Furthermore, it was predicted that about 23 of 1,385 FDA-approved non-oncology drugs would show efficacy against single diseases.

It was predicted that 4 drugs were active against various diseases.

The present invention relates to cancer-drug response scanning (CDRscan), which is used for a system and a method for drug indications and drug response and is a novel learning model capable of reliably predicting drug response by analyzing the convergence of specific genetic variation fingerprints associated with diseases, including cancers, and drug molecular pharmacophores. According to the present invention, the responsiveness of genomes to drugs whose pharmacological effects have not been found can be predicted from drug response data for genomes, which are collected from in vitro and in vivo clinical trials.

As described above, according to the present invention, the response of genomes to drugs whose pharmacological effects have not been found can be predicted from drug response data for genetic information, which are collected from in vivo and in vitro experiments or experiments on target proteins.

Namely, according to the present invention, the response correlation between drug pharmacophores and genomic variation information can be derived. Thus, when the genetic variations and drug pharmacophores to be analyzed are extracted, the response of drugs to the genome of interest can be reliably predicted.

Furthermore, according to the present invention, the response correlation between drug pharmacophores and genomic variation features can be derived. Thus, when the genetic variation features and drug pharmacophores to be analyzed are extracted, the response of the genome of interest to drugs can be reliably predicted.

Therefore, according to the present invention, the response of target proteins, cell lines or human bodies, which contain a particular genome, to unknown polymer compounds (substances to be developed as drugs), can be predicted prior to clinical trials. This can remarkably reduce the time and cost of the development of new drugs. In addition, the response of genomes other than genomes found in clinical trials to already developed drugs can be predicted. This can remarkably reduce research costs and time for the development of other applications and identification of side effects of existing drugs.

The scope of the present invention is not limited to the embodiments described above and should be defined by the claims. Those skilled in the art will appreciate that various changes and modifications are possible without departing from the scope of the present invention as defined by the appended claims.

1. A system of predicting drug indications and drug response using an artificial intelligence (AI) deep learning model based on convergence of different types of information, the system comprising:
a learning module configured to learn the response correlation between structure information on a drug and genetic information on a genome from collected learning information by deep machine learning;
a prediction module configured to receive analysis information and output the result of prediction of the response of the genome to the drug from the analysis information; and
a storage module configured to store a response prediction algorithm learned by the learning module,
wherein the learning information is drug response information obtained from clinical drug response information on target proteins, cell lines or living bodies.

2. The system of claim 1, wherein the learning module comprises:
a learning data generation unit configured to generate learning data for deep machine learning from the collected learning information;
a deep machine learning unit configured to perform deep machine learning for a plurality of learning data generated from the learning data generation unit; and
a response prediction algorithm generation unit configured to predict the response of the genome to the drug.

3. The system of claim 2, wherein the structure information is descriptor information on the drug.

4. The system of claim 3, wherein the drug is any one of nutrients, unknown unspecified drugs whose pharmacological mechanism is not known, or specified drugs whose pharmacological mechanism is known.

5. The system of claim 4, wherein the genetic information is mutation information on the genome.

6. The system of claim 5, wherein the learning data are a plurality of information that represent the response between a group of mutation information contained in the clinical information on the target proteins, cell lines or living bodies, and a group of descriptor information on the drug.

7. The system of claim 4, wherein the genetic information is feature information on mutations contained in the clinical information on the target proteins, cell lines or living bodies.

8. The system of claim 7, wherein the feature information comprises any one or more of mutability or entropy of variants, variant frequency in cancer, driver mutation score, 3D structure mutation environment, clinical significance mutation, drug response stratification attributable to genetic interaction, epigenomics, transcriptomics, or proteomics.

9. The system of claim 2, wherein the learning data are a plurality of information that represent the response between a group of feature information on mutations contained in the clinical information on the target proteins, cell lines or living bodies, and a group of descriptor information on the drug.

10. The system of claim 9, wherein the deep machine learning unit is configured to learn the response correlation between each genetic information contained in the clinical information on the target proteins, cell lines or living bodies, and each descriptor information on the drug, by deep machine learning for the learning data.

11. The system of claim 10, wherein the deep machine learning is performed by a Convolutional Neural Network (CNN) model.

12. The system of claim 10, wherein the deep machine learning is performed by a TensorFlow machine learning engine.

13. The system of claim 9, wherein the learning information is collected from:
target protein-drug dissociation constant, cancer cell line encyclopedia (CCLE); or
genomics of drug sensitivity in cancer (GDSC); or
clinical information databases for in vivo drug responses.

14. The system of claim 10, wherein the deep machine learning comprises the steps of:
(A1) collecting learning information which represents the response of each cell line genome to each drug;
(A2) generating genetic information on genomes from the learning information;
(A3) generating structure information from the learning information;
(A4) generating learning layers that represent the response between a group of the genetic information on the genomes and a group of the structure information on the drugs from the learning information; and
(A5) deriving the response correlation between individual genetic information and individual structure information by deep machine learning for the learning layers.

15. The system of claim 14, wherein the response is determined based on the dissociation constant of the target protein, the inhibition index IC₅₀ of the cell line, or clinical information (CR, PR, SD or PD) on in vivo drug response.

16. The system of claim 14, wherein the response prediction algorithm generation unit is configured to generate an algorithm that predicts the response between genetic information on the genome and structure information on the drug, through the response correlation between the genetic information and the structure information, learned by the deep machine learning unit.

17. The system of claim 16, wherein the prediction of drug response by the prediction module comprises the steps of:
(C1) receiving analysis information;
(C2) generating genetic information for analysis on genomes from the analysis information;
(C3) generating structure information for analysis on drugs from the analysis information; and
(C4) outputting the result of prediction of the response of the genome to the drug from the analysis information on the basis of the response correlation between the genomic information for analysis and the structure information for analysis by the response prediction algorithm.

18. The system of claim 17, wherein the structure information for analysis is descriptor information on the drug.

19. The system of claim 17, wherein the genetic information for analysis is mutation information on the genome.

20. The system of claim 17, wherein the genetic information for analysis is feature information on mutations contained in the genome.

21. The system of claim 16, wherein the prediction algorithm is configured to merge prediction values generated by different deep machine learning prediction algorithms.

22. The system of claim 21, wherein the different deep machine learning prediction algorithms are configured to calculate the weighted sum of hidden units of layers in which different types of feature information are merged, and then apply nonlinear function Relu, hyperbolic tangent or sigmoid function to the calculation results.

23. The system of claim 10, wherein the deep machine learning comprises the steps of:
(B1) collecting learning information that represents the response of each cell line genome to each drug;
(B2) generating genetic information on genomes contained in the learning information;
(B3) generating genetic information learning layers that represent the response between a group of the genetic information on each genome and the drug;
(B4) generating the response correlation between each genetic information and the drug by deep machine learning for the genetic information learning layers;
(B5) generating structure information on the drug contained in the learning information;
(B6) generating structure information learning layers that represent the response between each genome and a group of the structure information on the drug;
(B7) generating the response correlation between each genome and each structure information by deep machine learning for the structure information learning layers; and
(B8) generating the response correlation between individual genetic information and individual structure information through the response correlation between each genetic information and the drug, generated in step (B4), and the response correlation between each genome and each structure information, generated in step (B7).

24. The system of claim 23, wherein the response is determined based on the dissociation constant of the target protein, the inhibition index IC₅₀ of the cell line, or clinical information (CR, PR, SD or PD) on in vivo drug responses.

25. The system of claim 23, wherein the response prediction algorithm generation unit is configured to generate an algorithm that predicts the response between genetic information on the genome and structure information on the drug, through the response correlation between the genetic information and the structure information, learned by the deep machine learning unit.

26. The system of claim 25, wherein the prediction of drug response by the prediction module comprises the steps of:
(C1) receiving analysis information;
(C2) generating genetic information for analysis on genomes from the analysis information;
(C3) generating structure information for analysis on drugs from the analysis information; and
(C4) outputting the result of prediction of the response of the genome to the drug from the analysis information on the basis of the response correlation between the genomic information for analysis and the structure information for analysis by the response prediction algorithm.

27. The system of claim 26, wherein the structure information for analysis is descriptor information on the drug.

28. The system of claim 26, wherein the genetic information for analysis is mutation information on the genome.

29. The system of claim 26, wherein the genetic information for analysis is information on mutations contained in the genome.

30. The system of claim 25, wherein the prediction algorithm is configured to merge prediction values generated by different deep machine learning prediction algorithms.

31. The system of claim 30, wherein the different deep machine learning prediction algorithms are configured to calculate the weighted sum of hidden units of layers in which different types of feature information are merged, and then apply nonlinear function Relu, hyperbolic tangent or sigmoid function to the calculation results.


SEQUENCE LISTING (10 sequences)

SEQ ID NO: 1 - DNA, 35 bp, Artificial Sequence
  Synthetic nucleotide
gcaaggggtaacttccagcatggccacactgacgt

SEQ ID NO: 2 - DNA, 35 bp, Artificial Sequence
  Synthetic nucleotide
ggggtcatgctgccatgctgtggggttaagcctgc

SEQ ID NO: 3 - DNA, 37 bp, Artificial Sequence
  Synthetic nucleotide
tctggtaaagcaggtagcttttttcaggttattatag

SEQ ID NO: 4 - DNA, 35 bp, Artificial Sequence
  Synthetic nucleotide
gactgatcggtggggatggttgggatccttggatc

SEQ ID NO: 5 - DNA, 35 bp, Artificial Sequence
  Synthetic nucleotide
ccagctgcggggctcccactgccagccgctgagac

SEQ ID NO: 6 - protein, 41 aa, Artificial Sequence
  Synthetic peptide
GHEFVFLLKGHMQLFGLVNTLLANDPTSLRKNLSIQRACGT

SEQ ID NO: 7 - protein, 42 aa, Artificial Sequence
  Synthetic peptide
KTLKAGYTEKQMGQFSHHNIIRLEGVISKYKPMMIITGVISK

SEQ ID NO: 8 - protein, 41 aa, Artificial Sequence
  Synthetic peptide
KRVKDSDDVPLPTRTVDTKQAHELAKSYGIPFIETSAKDSD

SEQ ID NO: 9 - protein, 42 aa, Artificial Sequence
  Synthetic peptide
MTAIIKEIVSGFDLDLTYIYPNIIAMGFPAERLEGVYRLDLT

SEQ ID NO: 10 - protein, 41 aa, Artificial Sequence
  Synthetic peptide
DNPEYSPDPSILDLWQVKSGTIFDNFLITNDEAYAEEWQVK